(19) United States
(12) Patent Application Publication
(10) Pub. No.: US 2020/0170553 A1
(43) Pub. Date: Jun. 4, 2020

(54) MEASURING APPARATUS AND MEASURING METHOD

(71) Applicants: Ryosuke KASAHARA, Tokyo (JP); Yuji MATSUURA, Miyagi (JP)

(72) Inventors: Ryosuke KASAHARA, Tokyo (JP); Yuji MATSUURA, Miyagi (JP)

(21) Appl. No.: 16/640,152

(22) PCT Filed: Aug. 7, 2018
(86) PCT No.: PCT/JP2018/029666

§ 371 Date: Feb. 19, 2020

(30) Foreign Application Priority Data
Aug. 23, 2017 (JP) 2017-160481
May 23, 2018 (JP) 2018-099150

(51) Int. Cl. A61B 5/145; A61B 5/1455; A61B 5/00

(52) U.S. Cl. CPC A61B 5/14532; A61B 5/1455; A61B 2560/0238; A61B 2562/0233; A61B 5/7221

(57) ABSTRACT

A measuring apparatus includes a light source configured to output light in a mid-infrared region, a detector configured to irradiate a measuring object with the light output from the light source and detect reflected light reflected by the measuring object, and a blood glucose level measuring device configured to measure a blood glucose level of the measuring object. A wavenumber between a plurality of absorption peak wavenumbers of glucose is used as a blood glucose level measuring wavenumber for measuring the blood glucose level.

### TECHNICAL FIELD

The present invention relates to a noninvasive blood glucose level measurement technique.

### BACKGROUND ART

In recent years, diabetic patients are increasing worldwide, and noninvasive blood glucose measurement techniques that does not require blood sampling are becoming increasingly desirable. In this regard, various methods have been proposed including technologies that use radiation in the near-infrared or mid-infrared region and Raman spectroscopy. The methods using radiation in the mid-infrared region corresponding to a fingerprint region where glucose exhibits strong absorption are advantageous for improving measurement sensitivity as compared with methods using radiation in the near-infrared region.

A light emitting device such as a quantum cascade laser (QCL) can be used as a light source for emitting light in the mid-infrared region. However, in such case, the number of laser light sources is determined by the number of wavenumbers used. Thus, to achieve device miniaturization, the number of wavenumbers in the mid-infrared region used for measuring blood glucose levels is preferably reduced to no more than several wavenumbers.

A method has been proposed for accurately measuring glucose levels using radiation in the mid-infrared region by attenuated total reflection (ATR) by using wavenumbers corresponding to the absorption peaks of glucose (1035 cm⁻¹, 1080 cm⁻¹, 1110 cm⁻¹) (e.g., see Patent Document 1). Also, a method for creating a calibration model for non-invasive blood glucose measurement has been proposed (e.g., see, Patent Document 2).

### CITATION LIST

### Patent Literature

[PTL 1] Japanese Patent No. 5376439

[PTL 2] Japanese Patent No. 4672147

### SUMMARY OF INVENTION

### Technical Problem

In developing practical applications of noninvasive blood glucose measurement techniques, measurement robustness with respect to various conditions and environmental changes and measurement reliability are particularly important. However, with measurement techniques using glucose absorption peak wavenumbers, securing robustness with respect to influences of other metabolites and changes in measurement conditions has been a challenge.

An aspect of the present invention is to directed to providing a noninvasive blood glucose level measuring apparatus and a measuring method having high measurement reliability and environmental robustness.

### Solution to Problem

According to one aspect of the present invention, a measuring apparatus includes a light source configured to output light in a mid-infrared region, a detector configured to irradiate a measuring object with the light output from the light source and detect reflected light reflected by the measuring object, and a blood glucose level measuring device configured to measure a blood glucose level of the measuring object. A wavenumber between a plurality of absorption peak wavenumbers of glucose is used as a blood glucose level measuring wavenumber for measuring the blood glucose level.

### Advantageous Effects of Invention

According to one aspect of the present invention, blood glucose level measurement with high measurement reliability and environmental robustness may be implemented.

### BRIEF DESCRIPTION OF DRAWINGS

FIG. 1A is a schematic diagram of a measuring apparatus implementing an aspect of the present invention.

FIG. 1B is a schematic diagram of an ATR prism used in the measuring apparatus.

FIG. 2A is a schematic diagram of a measuring apparatus according to an embodiment of the present invention.

FIG. 2B is a schematic diagram of an ATR prism used in the measuring apparatus according to an embodiment of the present invention.

FIG. 2C is a schematic diagram of a hollow optical fiber used in the measuring apparatus according to an embodiment of the present invention.

FIG. 3 is a table indicating datasets used in an embodiment of the present invention.

FIG. 4 is a flowchart illustrating a wavenumber selection process.

FIG. 5 is a graph representing example interpolations of blood glucose levels immediately after measurement and after the lapse of a fixed time period.

FIG. 6 is a comparison diagram illustrating the difference between a general leave-one-out cross validation and a series cross validation used in an embodiment of the present invention.

FIG. 7A is a graph representing the absorption spectrum of dataset 1.

FIG. 7B is a graph representing the absorption spectrum of dataset 2.

FIG. 8A is a graph representing a correlation coefficient map for the delay and the number of wavenumbers in series cross validation.

FIG. 8B is a graph representing a correlation coefficient map for the delay and the number of components in series cross validation.

FIG. 9 is a histogram representing the number of wavenumbers selected as a function of the wavenumber and delay.

FIG. 10 is a graph representing the correlation coefficient as a function of time delay for selected wavenumbers and glucose absorption peak wavenumbers.

FIG. 11A is a Clarke error grid for dataset 1 in the multiple linear regression model.

FIG. 11B is a Clarke error grid for dataset 1 in the PLS model.

FIG. 12A is a Clarke error grid for dataset 2 in the multiple linear regression model.

FIG. 12B is a Clarke error grid for dataset 2 in the PLS model.

FIG. 13 is a schematic diagram illustrating a case where there is a space between an ATR prism and a measurement surface.

FIG. 14 is a mapping of the coefficient of determination when two wavenumbers are selected and the time delay is 0 minutes.

FIG. 15 is a mapping of the coefficient of determination when two wavenumbers are selected and the time delay is 10 minutes.

FIG. 16 is a mapping of the coefficient of determination when two wavenumbers are selected and the time delay is 20 minutes.

FIG. 17 is a mapping of the coefficient of determination when two wavenumbers are selected and the time delay is 30 minutes.

FIG. 18 is a mapping of the coefficient of determination when two wavenumbers are selected and the time delay is 40 minutes.

FIG. 19 is a mapping of the coefficient of determination when two wavenumbers are selected and the time delay is 20 minutes across a wider wavenumber range.

FIG. 20 is a graph representing changes in the coefficient of determination as a function of the combination of candidate wavenumbers and the time delay.

FIG. 21 is a graph representing changes in the coefficient of determination as a function of the combination of candidate wavenumbers and the time delay.

FIG. 22 is a graph representing changes in the regression coefficients as a function of the time delay when two wavenumbers are selected from candidate wavenumbers.

FIG. 23 is a graph representing changes in the regression coefficients as a function of the time delay when two wavenumbers are selected from the candidate wavenumbers.

FIG. 24 is a graph representing changes in the regression coefficients as a function of the time delay when two wavenumbers are selected from the candidate wavenumbers.

FIG. 25 is a diagram illustrating a part of the glycolysis pathway.

FIG. 26 is a graph representing an infrared ATR absorption spectrum of an aqueous glucose solution and a whole blood sample.

FIG. 27 is a graph representing the absorption spectrum of each substance and the wavenumbers selected in the embodiment.

FIG. 28 is a graph indicating the sensitivity to each substance when two wavenumbers are selected.

FIG. 29 is a graph indicating the sensitivity to each substance when two wavenumbers are selected.

FIG. 30 is a graph indicting the sensitivity to each substance when two wavenumbers are selected.

FIG. 31 is a graph representing a tolerance evaluation of a selected wavenumber when a coefficient of determination is adjusted according to a wavenumber shift.

FIG. 32 is a graph representing a tolerance evaluation of a selected wavenumber when a coefficient of determination is adjusted according to a wavenumber shift.

FIG. 33 is a graph representing a tolerance evaluation of a selected wavenumber when a coefficient of determination is adjusted according to a wavenumber shift.

FIG. 34 is a graph representing a tolerance evaluation of a selected wavenumber when the coefficient of determination is fixed.

FIG. 35 is a graph representing a tolerance evaluation of a selected wavenumber when the coefficient of determination is fixed.

FIG. 36 is a graph representing a tolerance evaluation of a selected wavenumber when the coefficient of determination is fixed.

FIG. 37 is a graph indicating abnormality detection of blood glucose level measurement.

FIG. 38 is a table indicating the coefficient of determination of blood glucose level regression when one wavenumber is excluded from the three wavenumbers used in the embodiment.

FIG. 39 is a diagram illustrating a modified example of the measuring apparatus.

FIG. 40 is a functional block diagram of an information processing apparatus that performs noninvasive calibration using the measuring apparatus according to an embodiment of the present invention.

FIG. 41 is a flowchart illustrating a process of learning and evaluation of a prediction result.

FIG. 42 is a diagram illustrating training data and test data used in the process of FIG. 41.

FIG. 43 is a network diagram used in a calibrator according to an embodiment of the present invention.

FIG. 44 is a flowchart illustrating a learning process implemented in the network of FIG. 43.

FIG. 45 is a graph showing changes in the loss for each step in a model learning process.

FIG. 46A is graph representing a data distribution of a representative series of dataset 2 without domain adaptation.

FIG. 46B is graph representing a data distribution of a representative series of dataset 2 with domain adaptation.

FIG. 47A is a Clarke error grid for a prediction model obtained without domain adaptation.

FIG. 47B is a Clarke error grid for a prediction model obtained with domain adaptation.

FIG. 48 is a table comparing the correlation coefficient and the ratio of data points in region A of the Clarke error grid for various models.

FIG. 49 is a graph showing the influence of noise on the correlation coefficient for dataset 1.

FIG. 50 is a graph showing the influence of noise on the correlation coefficient for dataset 2.

### DESCRIPTION OF EMBODIMENTS

In the following, embodiments of the present invention will be described with reference to the accompanying drawings.

In order to implement noninvasive blood glucose measurement with high reliability and robustness, embodiments of the present invention are directed to the following aspects:

(1) finding a small number of wavenumbers suitable for noninvasive blood glucose measurement in the mid-infrared region, and

(2) building a robust prediction model that can accommodate a wide range of individual differences, measurement environment difference, and the like.

With regard to the first aspect relating to wavenumber selection, a mid-infrared spectrometer is expensive and requires cooling. Thus, considering the cost and device configuration, a laser light source such as QCL is preferably used, and the number of wavenumbers to be used is preferably reduced to several wavenumbers. In wavenumber selection, a wavenumber that can improve the blood glucose level measurement accuracy is selected in consideration of the absorbance of glucose as well as other substances that can be simultaneously measured and metabolic substances in the body.

In embodiments of the present invention, instead of using glucose absorption peak wavenumbers that are generally used, a wavenumber other than the glucose absorption peak wavenumber is used as a blood glucose level measuring wavenumber. For example, a wavenumber between one absorption peak and another absorption peak of glucose may be used. For example, assuming k denotes a wavenumber in the mid-infrared region, one or more blood glucose level measuring wavenumbers may be selected from a wavenumber range of 1035 cm⁻¹<k<1080 cm⁻¹ and/or a wavenumber range of 1080 cm⁻¹<k<1100 cm⁻¹. Preferably, the number of wavenumbers used is less than or equal to three. In addition to using one or more blood glucose level measuring wavenumbers, a wavenumber other than the blood glucose level measuring wavenumbers may be used to estimate a reliability of measurement, for example.

With regard to the second aspect relating to building a prediction model with high environmental robustness, many variable factors affect the accuracy of noninvasive blood glucose measurement, such as the difference in meal content, physical differences between individual patients, and environmental variations at the time of measurement. Unless a robust prediction model that accommodates such factors can be built, practical application of a noninvasive blood glucose measurement technique may be difficult. In embodiments of the present invention, instead of using the leave-one-out cross validation (LOOCV), which is generally used as a verification method for a prediction model, a more stringent cross validation is used, in which a data group including a series of post-meal measurements performed at the same occasion is not used for model estimation and accuracy verification at the same time (different series of data groups are used for model estimation and accuracy verification). Such cross validation used in embodiments of the present invention is hereinafter referred to as “series cross validation”.

By selecting a wavenumber in the mid-infrared region based on a prediction model implementing series cross validation, measurement that is less dependent on a specific environment or specific data may be enabled. As described below, by using a prediction model according to an embodiment of the present invention, measurement may be performed using three wavenumbers or two wavenumbers in the mid-infrared region, and the accuracy of the measurement may be comparable to the case of performing multi-wavenumber measurement using at least several dozen wavenumbers, for example. Also, by using a prediction model implementing series cross validation, correlation can be obtained without performing calibration with respect to data obtained at different dates/times, different seasons, different subjects, different meals, and different devices, for example.

Further, by applying neural network using adversarial training in domain adaptation (DANN: Domain Adversarial Neural Network) to blood glucose measurement, calibration without blood sampling may be enabled.

<Apparatus Configuration>

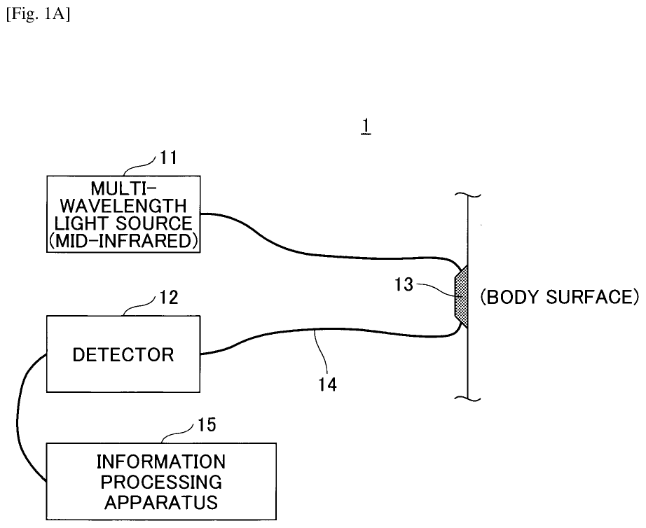

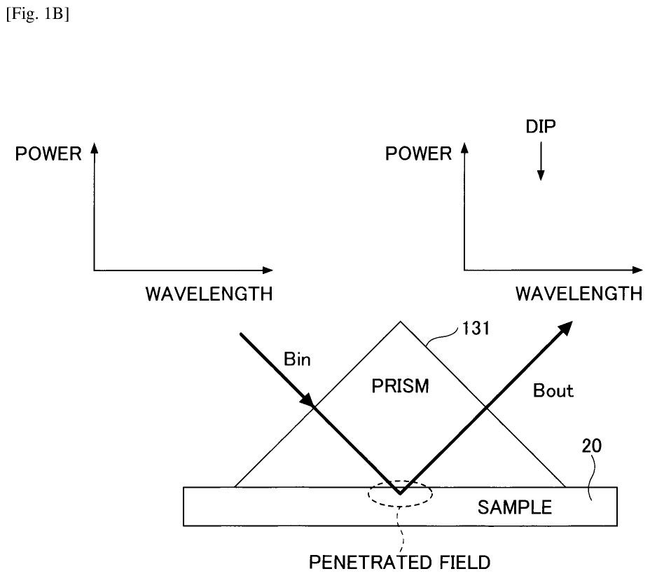

FIG. 1A is a schematic diagram of a measuring apparatus 1 to which the present invention is applied. In FIG. 1A, the measuring apparatus 1 includes a multi-wavelength light source 11, an optical head 13 including an ATR prism 131, a detector 12, and an information processing apparatus 15. The multi-wavelength light source 11, the optical head 13, and the detector 12 are connected to each other by an optical fiber 14. The mid-infrared light emitted from the multi-wavelength light source 11 is irradiated onto a measuring object (e.g., body surface such as skin, lip, or the like) via the optical fiber 14 and the optical head 13.

As illustrated in FIG. 1B, the ATR prism 131 of the optical head 13 is placed in contact with a sample 20 to be measured. At the ATR prism 131, the infrared light undergoes attenuation corresponding to the infrared absorption spectrum of the measuring object. The attenuated light is received by the detector 12, and the intensity for each wavenumber is measured. The measurement results are input to the information processing apparatus 15. The information processing apparatus 15 analyzes the measurement data and outputs the blood sugar level and the measurement reliability.

The infrared attenuated total reflection (ATR) method is effective for spectroscopic detection in the mid-infrared region where strong glucose absorption can be obtained. In the infrared ATR method, infrared light is incident on the ATR prism 131 with a high refractive index and the “penetrated field” that occurs when total reflection occurs at the boundary surface between the prism and the exterior (e.g., sample) is used. If the measurement is performed while the sample 20 to be measured is in contact with the ATR prism 131, the penetrated field is absorbed by the sample 20.

When light from an infrared lamp having a wide wavelength range of 2-12 μm is used as the incident light, light at a relevant wavelength according to the molecular vibration energy of the sample 20 is absorbed, and the light absorption at the relevant wavelength of the light transmitted through the ATR prism 131 appears as a dip. In this method, detected light transmitted through the ATR prism 131 may not sustain substantial energy loss such that it is particularly advantageous in infrared spectroscopy using lamp light with weak power.

When infrared light is used, the penetration depth of light from the ATR prism 131 to the sample 20 is only about several microns such that the light does not reach capillaries, which exist at depths of about several hundred microns. However, components such as plasma in blood vessels leak out as tissue fluid (interstitial fluid) into skin and mucosal cells. By detecting the glucose component present in such tissue fluid, the blood glucose level can be measured.

The concentration of glucose components in interstitial fluid is assumed to increase at depths closer to the capillary, and as such, the ATR prism 131 is always pressed against a sample with a constant pressure at the time of measurement. In this respect, in embodiments of the present invention, a multiple reflection ATR prism having a trapezoidal cross section is used.

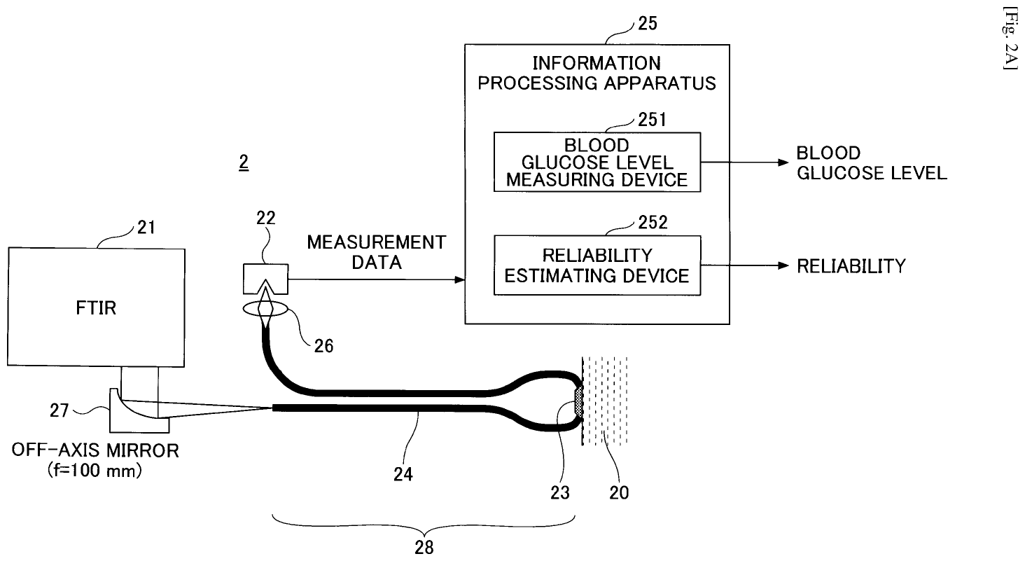

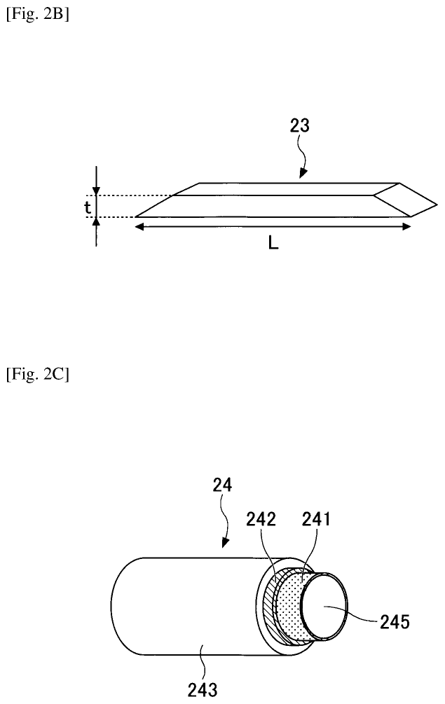

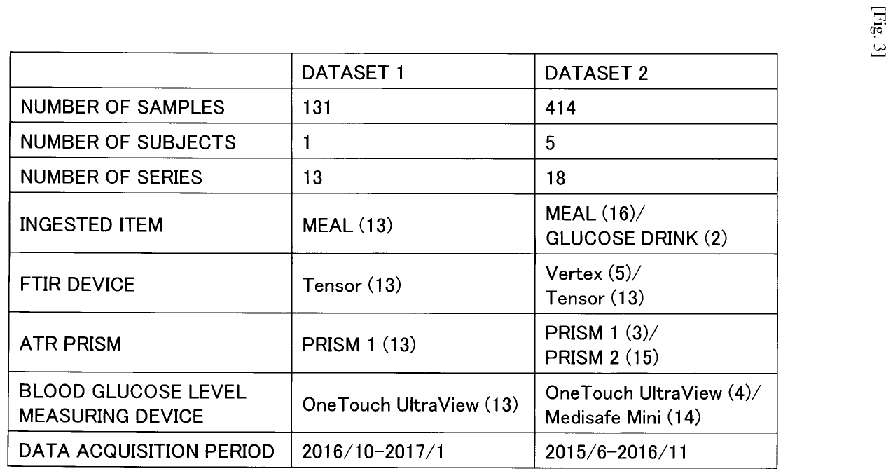

FIG. 2A is a schematic diagram of a measuring apparatus 2 according to an embodiment of the present invention. In FIG. 2A, the measuring apparatus 2 includes a Fourier transform infrared spectroscopy (FTIR) device 21, an ATR probe 28 including an ATR prism 23, a detector 22, and an information processing apparatus 25. Infrared light output from the FTIR device 21 is incident on a hollow optical fiber 24 by an off-axis parabolic mirror 27 and undergoes attenuation corresponding to the infrared light absorption spectrum of the sample 20 at the ATR prism 23. The attenuated light that has passed through the hollow optical fiber 24 and the lens 26 is detected by the detector 22. The detection result is input to the information processing apparatus 25 as measurement data.

The information processing apparatus 25 includes a blood sugar level measuring device 251 and a reliability estimating device 252. The blood glucose level measuring device 251 measures a blood glucose level based on measurement data (infrared light spectrum) using a prediction model as described below and outputs the blood glucose level measurement. Note that the blood glucose level measuring device 251 is an example of a blood sugar level measuring device according to the present invention. The reliability estimating device 252 calculates the measurement reliability using a wavenumber different from the wavenumber used for blood glucose level measurement, for example, and outputs the calculated measurement reliability as described below.

The measuring apparatus 2 uses several wavenumbers for blood glucose measurement, and the wavenumbers are selected from a range between one absorption peak and another absorption peak of glucose. For example, an absorption spectrum for wavenumbers 1050±6 cm⁻¹, 1070±6 cm⁻¹, and 1100±6 cm⁻¹ may be used.

As illustrated in FIG. 2B, the ATR prism 23 is a trapezoid prism. The glucose detection sensitivity may be enhanced by multiple reflections at the ATR prism 23. Also, the ATR prism 23 can secure a relatively large contact area with the sample 20 such that fluctuations in detection values due to changes in the pressure of the ATR prism 23 pressing against the sample 20 may be reduced. The bottom face of the ATR prism 23 may have a length L of 24 mm, for example. The ATR prism 23 is arranged to be relatively thin to enable multiple reflections, and for example, its thickness t may be set to 1.6 mm, 2.4 mm, or the like.

Potential materials of the prism include materials that are not toxic to the human body and exhibit high transmission characteristics around the wavelength of 10 μm corresponding to the absorption band of glucose that is being measured. In the present embodiment, a prism made of ZnS (zinc sulfide), which has a low refractive index (refractive index: 2.2) and high penetration to enable detection at greater depths, is used. Unlike ZnSe (zinc selenide), which is commonly used as an infrared material, ZnS (zinc sulfide) is known to be free of carcinogenic properties and is also used for dental materials as a non-toxic dye (lithopone).

In general ATR measuring apparatuses, the prism is fixed in a rather bulky housing such that an area to be measured is usually limited to skin surfaces such as the fingertip or the forearm. However, these skin areas are covered by thick stratum corneum with a thickness of about 20 μm, and as such, the detected glucose component concentration tends to be low. Also, measurement of the stratum corneum is affected by secretion of sweat and sebum, for example, such that measurement reproducibility is limited. In this respect, the measuring apparatus 2 according to the present embodiment uses the hollow optical fiber 24 that is capable of transmitting infrared light with low loss, and the ATR probe 28 having the ATR prism 23 attached to the tip of the hollow optical fiber 24. By using the ATR probe 28, measurements may be made at the ear lobe, which has capillary vessels located relatively close to the skin surface and is less susceptible to influences of sweat and sebum, or the oral mucosa having no keratinized layer, for example.

FIG. 2C is a schematic diagram of the hollow optical fiber 24 used in the measuring apparatus 2. Mid-infrared light having a relatively long wavelength that is used for glucose measurement is absorbed by glass and cannot be transmitted through an ordinary quartz glass optical fiber. Although various types of optical fibers for infrared transmission using special materials have been developed, these materials have not been suitable for medical use due to issues with toxicity, hygroscopicity, chemical durability, and the like. On the other hand, the hollow optical fiber 24 has a metal thin film 242 and a dielectric thin film 241 arranged in the above recited order around an inner surface of a tube 243 made of harmless material such as glass or plastic. The metal thin film 242 is made of a material having low toxicity such as silver and is coated with the dielectric thin film 241 to provide chemical and mechanical durability. Also, the hollow optical fiber 24 has a core 245 formed by air that does not absorb mid-infrared light, and in this way, the hollow optical fiber 24 is capable of low-loss transmission of mid-infrared light in a wide wavelength range.

<Demonstration Experiment>

Using the measuring apparatus 2 of FIG. 2, the absorbance of the oral mucosa is measured. As described above, the measuring apparatus 2 uses, as a transmission line, the hollow optical fiber 24 that is capable of efficiently propagating mid-infrared light to the lips with little toxicity. “Tensor” and “Vertex” manufactured by Bruker Corporation are used as the FTIR device 21. As the ATR prism 23, two types of prisms including prism 1 having a thickness (t) of 1.6 mm and prism 2 having a thickness (t) of 2.4 mm are used. The length L of the bottom surfaces of the prisms are both 24 mm. The thinner prism 1 (t=1.6 mm) can promote more light reflection inside the ATR prism 23 and has higher sensitivity as compared with the prism 2 (t=2.4 mm).

In order to measure the blood glucose level in blood to be used as a reference, blood sampling is performed using a commercially available blood glucose level self-measuring device. “Medisafe Mini (registered trademark)” manufactured by Terumo Corporation and “One Touch UltraView (registered trademark)” manufactured by Johnson & Johnson Company are used as the self-measuring devices. Because there are deviations in blood glucose levels indicated for the same blood sample between these two self-measuring devices, the measurement value of “Medisafe Mini” is corrected by a linear expression to match the measurement value of “One Touch Ultra View”.

As a basic measurement method for data acquisition, measurement is started after a meal and the measurement is continued intermittently until the blood sugar level settles about 3 hours after the meal. During the measurement over a period of about 3 hours, blood glucose level measurement by blood sampling using the commercially available measuring device and optical noninvasive blood glucose level measurement according to an embodiment of the present invention were performed several to a dozen times, and the measurement results (blood glucose level in blood and spectrum information) are recorded. A series of data acquired at the same measurement occasion is hereinafter referred to as “data series”.

FIG. 3 is a table indicating characteristics of dataset 1 and dataset 2 obtained by the measurement. The characteristics include the number of samples (data points), the number of subjects, the number of data series, the ingested item, the type of FTIR device 21, the type of ATR prism 23, the type of self-measuring device, and the data acquisition period.

Dataset 1 contains 131 data points from 13 series of measurements performed over a period of five months on one healthy adult who was required to take various meals before the measurements. Dataset 2 contains 414 data points from 18 series of measurements performed over a period of 15 months on five healthy adults (different from the subject of dataset 1) who were required to take various meals or a glucose drink before the measurements. The glucose drink contained 75 g of glucose dissolved in 150 ml of water. Dataset 2 includes data acquired using different ATR prisms and different FTIR devices.

Using dataset 1 and dataset 2, mid-infrared wavenumbers to be used in blood glucose level measurement are searched and a prediction model is constructed for verification. First, using series cross validation for dataset 1 obtained from one single subject, correlated wavenumbers are extracted and a prediction model is constructed. Next, using the model created based on dataset 1, a determination is made as to whether prediction results for the data of dataset 2 are correlated with the blood glucose levels. The data of dataset 2 differ from those of dataset 1 in terms of the season in which they were acquired, the subjects, the meals, and the measuring devices used. Therefore, if correlations are found with dataset 2, using the prediction model constructed using dataset 1, it can be concluded that robust blood glucose measurement independent of various conditions can be achieved.

PLS (Partial Least Square) regression, SVM (Support Vector Machine), NN (Neural Network) and the like are known as models that regress measured spectrum data to blood glucose levels. In the embodiment, as a regression model of blood glucose level, a simple multiple linear regression (MLR) model with few parameters and less overfit is used to avoid deterioration of robustness due to overfit. The prediction model is expressed by equation (1). In the present embodiment, a simple multiple linear regression (MLR) model is used as the blood glucose level regression model. MLR has a small number of parameters and avoids overfitting to specific conditions or data which may lead to a degradation in robustness. The prediction model is represented by the following equation (1).

[Math.1]

y=Ax (1)

In the above equation (1), y represents the predicted blood glucose concentration, x represents the measured absorbance spectrum data, and A represents a regression model with sparse coefficients.

The problem to be solved to obtain the prediction model is represented by the following equation (2).

$$\begin{matrix}[ Math . 2 ] & \\ {min}_{x} {‖ y - Ax ‖}^{2} subject to {‖ x ‖}_{0} = L & ( 2 )\end{matrix}$$

In the above equation (2), L represents the number of wavenumbers to be used. The model optimization problem is to find a sparse regression model A that minimizes the least-squares error when the number of wavenumbers is limited.

In the present embodiment, it is assumed that the number of wavenumbers L ranges from 1 to 3, and for model optimization, searches are made for combinations of all wavenumbers for each value of L (number of wavenumbers), such that the least-squares error is minimized with respect to each series of series cross validation. Note that the above method is described in detail below. Also, for reference, the results of the MLR method using a few wavenumbers are compared with those obtained from PLS regression using a larger number of wavenumbers, which is generally used as a spectrum analysis and regression model for blood glucose levels. The above comparison is also described in detail below.

<Wavenumber Selection Process>

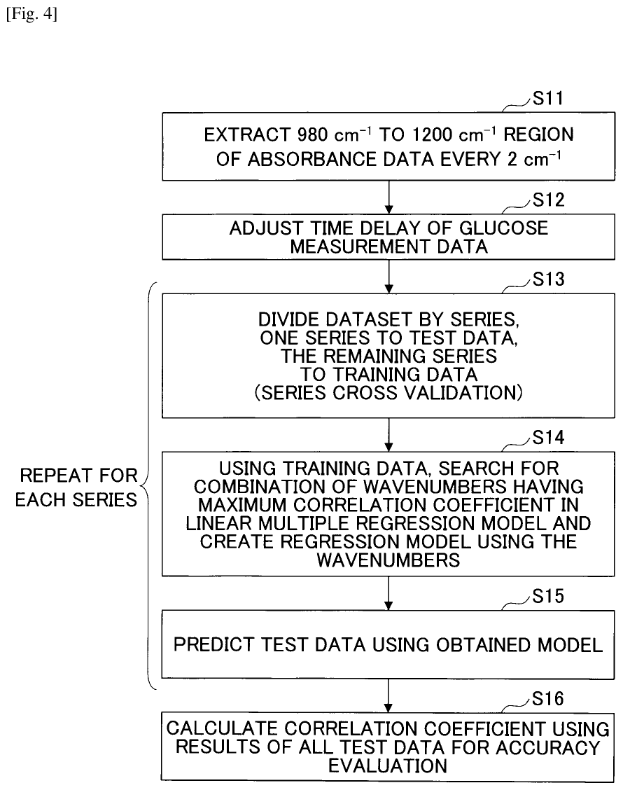

FIG. 4 is a flowchart illustrating a wavenumber selection process. First, a part of absorbance data x obtained by the FTIR device 21 corresponding to a region from 980 cm⁻¹ to 1200 cm⁻¹ where the absorption spectrum of glucose exists is extracted (interpolated) every 2 cm⁻¹ to generate spectrum information (step S11). Note that in creating datasets 1 and 2, samples that are obviously abnormal measurements as can be perceived from the spectrum data are deleted.

Next, the time delay of the glucose measurement data is adjusted (step S12). It takes more time for the glucose level in tissue fluid or intracellular metabolic system to reach the value of the blood glucose level in blood vessels. Therefore, the effect of this delay on the regression accuracy is examined by delaying the time of data acquisition of the blood glucose level relative to the data acquisition time of the corresponding spectrum, from 0 min to 40 min in increments of 2 min. Specifically, linear interpolation is applied to blood glucose levels measured at the time of mid-infrared light spectrum measurement to obtain blood glucose levels at respective times.

Assuming the initial blood glucose measurement time after a meal is set to “0 min”, blood glucose levels below “0 min” are interpolated to the blood glucose level at “0 min”, because the blood glucose level during fasting is considered invariant.

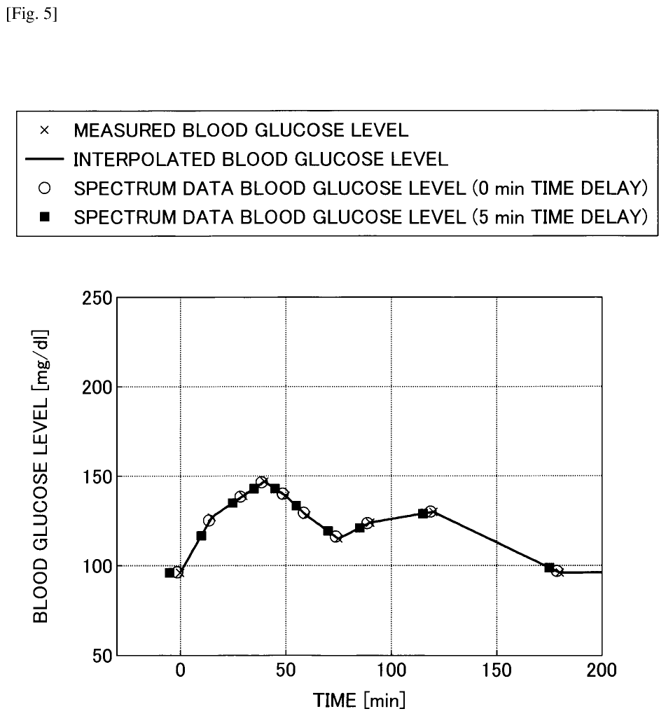

FIG. 5 illustrates an example blood glucose level interpolation result for time delays of 0 min and 5 min. In FIG. 5, the cross mark (×) indicates the blood glucose level in blood measured by the self-measuring device after a meal, the solid line indicates the linearly interpolated blood glucose level, the circle mark (◯) indicates the blood glucose level of the mid-infrared light spectrum with a time delay of “0 min”, and the square mark indicates the blood glucose level of the mid-infrared light spectrum with a time delay of “5 min”. Such time delay setting is performed for each data point. Note that for dataset 2, in order to remove the influence of the difference in the number of reflections of the two types of ATR prisms 23, the spectrum is normalized with respect to the wavenumber 1000 cm^{?1} corresponding to a dip in the absorption spectrum for glucose.

Referring back to FIG. 4, the dataset is divided for each series to perform series cross validation (step S13). In series cross validation, one data series is used as test data, and the remaining data series are used as training data. Each series includes multiple data points acquired at the same occasion.

In the common leave-one-out cross validation, one point in a dataset is used as test data, and the remaining points are used as training data for prediction model generation. A prediction model is created using the training data, and the precision of the test data is verified. Thus, assuming one series relates to a change in the blood glucose level of one subject after taking a certain meal, the training data and test data will contain data within the same series. It is easy to predict blood glucose levels in situations where the meal is the same. Therefore, even if required accuracy is obtained by leave-one-out cross validation using measurement data points of the same series as training data, accuracy may not necessarily be achieved with respect data acquired under different conditions (different meals) such as the dataset of the present embodiment in which a different meal is taken in each series. Also, even if a wavenumber with high correlation is selected using leave-one-out cross validation, the wavenumber may not necessarily be appropriate for general situations.

In contrast, series cross validation is a method in which only one series out of all data is used as test data, and all the remaining series are used as training data. The verification using series cross validation is more stringent than the verification using the leave-one-out cross validation, and it produces results that are closer to actual situations.

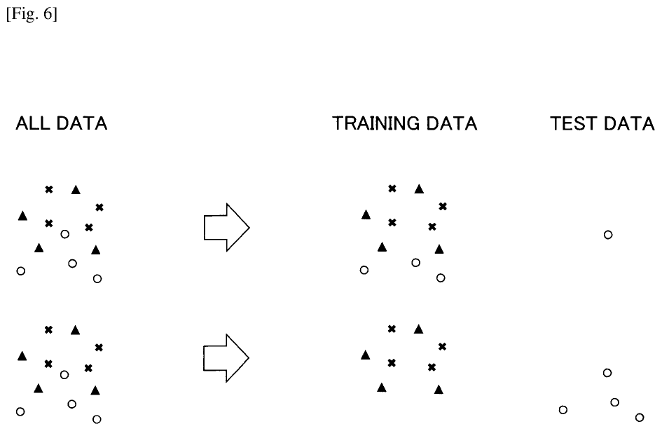

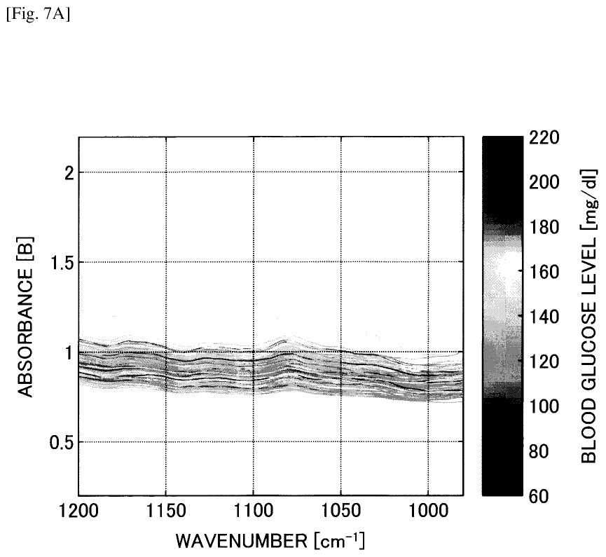

FIG. 6 is a schematic diagram comparing the principles of leave-one-out cross validation and series cross validation. In FIG. 6, leave-one-out cross validation is illustrated at the top, and series cross validation is illustrated at the bottom. The points indicate samples and their various shapes indicate different series. In leave-one-out cross validation, only one data point is used as test data, whereas in series cross validation, all data points included in a given series are used as test data. If high accuracy is achieved in series cross validation, over-fitting to the training data will be unlikely and prediction accuracy will more likely be ensured even if unknown data are present. However, because series cross validation is more stringent then leave-one-out cross validation, the correlation values (e.g. correlation coefficient) of test results will likely be lower.

Referring back to FIG. 4, using the training data, all combinations of wavenumbers are searched to find a combination of wavenumbers that will maximize the correlation coefficient in a multiple linear regression model, and a regression model is created using the combination of wavenumbers (step S14). Using the obtained regression model, test data is predicted (step S15). The prediction model y using the multiple linear regression model A is represented by the above equation (1).

Steps S13 to S15 are repeated for each data series. When all the test data are predicted, the correlation coefficient is calculated by combining the prediction results of all the data series and accuracy evaluation is performed (step S16).

In this wavelength selection process, wavenumbers that provide good verification results in series cross validation are selected so that a robust prediction model that can accommodate various measurement conditions and environmental conditions can be obtained. Also, by reducing the number of wavenumbers to a small number, prediction can be made with a minimum amount of data, generalization performance can be improved, and environmental robustness can be secured.

<Experimental Results>

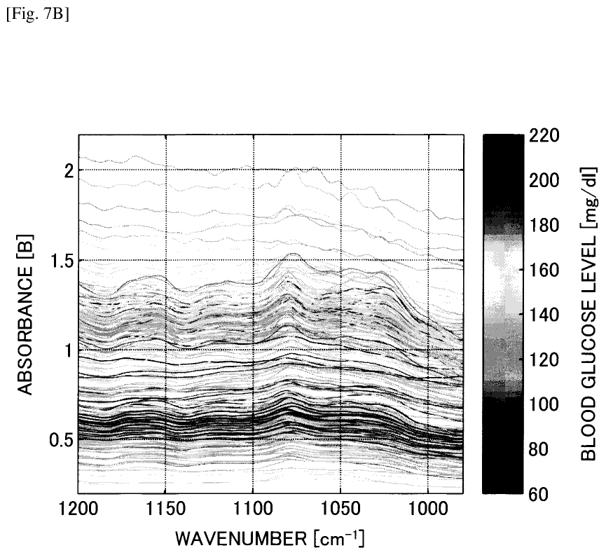

FIGS. 7A and 7B are graphs respectively indicating the absorption spectrum data of dataset 1 and dataset 2 generated in step S11. The vertical axis represents the absorbance, and the horizontal axis represents the wavenumber. Note that the spectrum data shown in FIGS. 7A and 7B are not normalized. The gradation bar at the right side of FIGS. 7A and 7B show the blood glucose level when the time delay is 0 minutes (i.e., at the time of first measurement after meal). Because dataset 1 is measurement data obtained using the same device for the same subject, the spectrum data of dataset 1 is consistent. Because dataset 2 includes measurement data obtained under various conditions, the spectrum data of dataset 2 has greater variation than that of dataset 1. However, the spectrum data of dataset 2 shows peaks at certain wavenumbers. Note that a dip appears in the spectrum data of dataset 2 at wavenumber 1000 cm⁻¹ and this wavenumber is used for normalization of dataset 2.

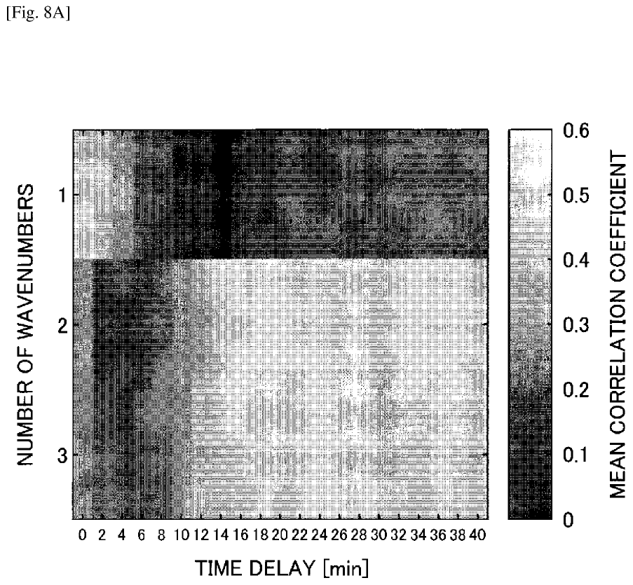

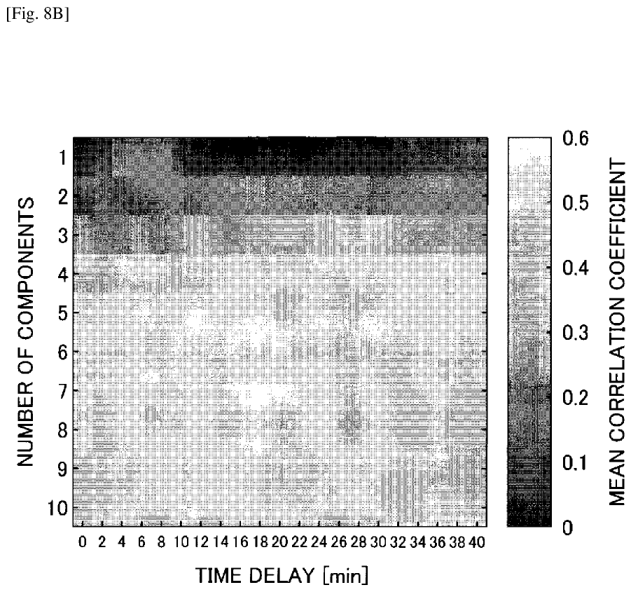

FIG. 8A shows a correlation coefficient map for the time delay and the number of features (the number of wavenumbers) in the multiple linear regression model A when implementing series cross validation in step S14. The number of wavenumbers is 1 to 3. The gradation bar at the right side shows the correlation coefficient. The greater the correlation coefficient, the lighter the gradation color. As can be appreciated from FIG. 8A, a region where the time delay is from 20 to 30 minutes and the number of wavenumbers is 2 to 3 has a large correlation coefficient. The correlation coefficient is maximized when the time delay is 26 minutes and the number of wavenumbers is 3. The correlation coefficient at this time is 0.49. Note that the absence of a large correlation at a time delay of 0 minutes indicates that it takes some time for a change in the blood glucose level in blood to be reflected in the infrared spectrum.

FIG. 8B shows a correlation coefficient map for the time delay and the number of features (number of components) in the PLS model when implementing series cross validation. In the PLS model, the number of components, as the number of features, is set to range from 1 to 10. It can be appreciated that the correlation coefficient becomes large in a region where the number of components is between 4 and 7 and the time delay is about 20 minutes. The correlation coefficient reaches its maximum value when the number of components is 6 and the time delay is 20 minutes, and the correlation coefficient at this time is 0.51. Note that one component of the PLS model includes components of all input wavenumbers (absorbance data extracted every 2 cm⁻¹ from the 980 cm⁻¹ to 1200 cm⁻¹ region). That is, even one component contains information of several hundred wavenumbers.

It can be appreciated from the above results that even when the number of selected wavenumbers is reduced to three wavenumbers, a correlation comparable to the case of selecting a large number of wavenumbers in the PLS model can be obtained. In the PLS model, even though a large number of wavenumbers are used, a minimum number and an optimum wavenumber cannot be selected. In the blood glucose level measurement using mid-infrared light according to the present embodiment, by only using 2 to 3 wavenumbers, the same level of measurement accuracy as that when using a substantially larger number of wavenumbers can be obtained.

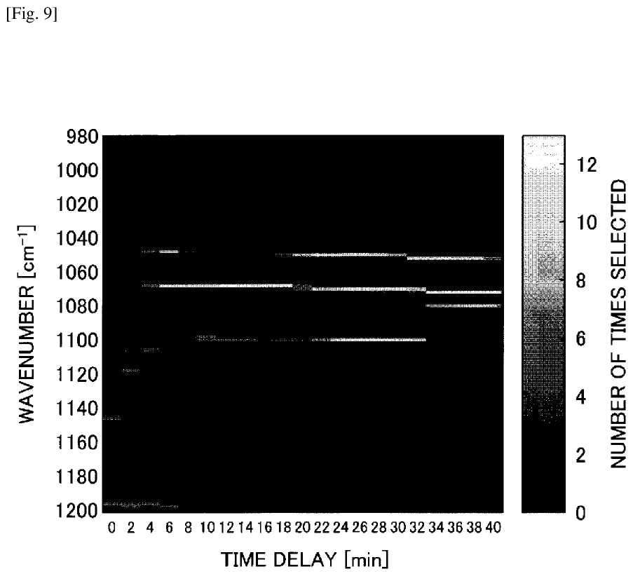

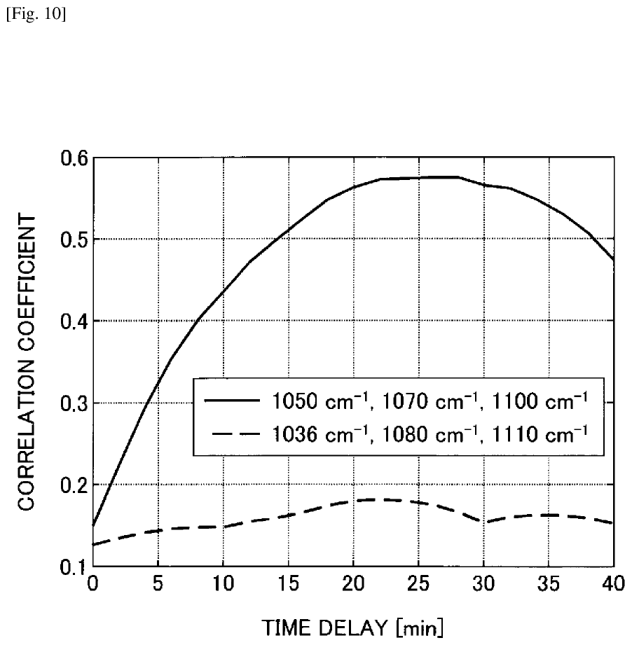

FIG. 9 is a histogram showing the number of times each wavenumber (or wavelength) is selected at different time delays in each data series in the case where the number of wavenumbers is set to L=3 (i.e., three wavenumbers are selected) in the multiple linear regression model A. The data series is data of each series used for series cross validation. It can be appreciated that there is little variation in the selected wavenumbers, and in the high correlation region where the time delay is from 20 to 30 minutes, wavenumbers of approximately 1050 cm⁻¹ (±several cm'), approximately 1070 cm¹ (±several cm¹), and approximately 1100 cm¹ (±several cm¹) are selected. Also, the selected wavenumbers vary depending on the time delay, thereby suggesting that the wavenumber suitable for blood glucose level mid-infrared spectrum measurement changes in relation to changes associated with metabolism in the body.

Note that the wavenumbers of 1050 cm⁻¹ (±several cm⁻¹), 1070 cm⁻¹ (±several cm⁻¹), and 1100 cm⁻¹ (±several cm⁻¹) are in the glucose fingerprint regions but they do not correspond to glucose absorption peaks. When the absorption peaks of glucose are simply used for in vivo measurement, it may be difficult to obtain correlation with blood glucose level due to interference of other substances. That is, it is highly likely that the measurement represents absorption of other substances in the body and metabolites of glucose, for example.

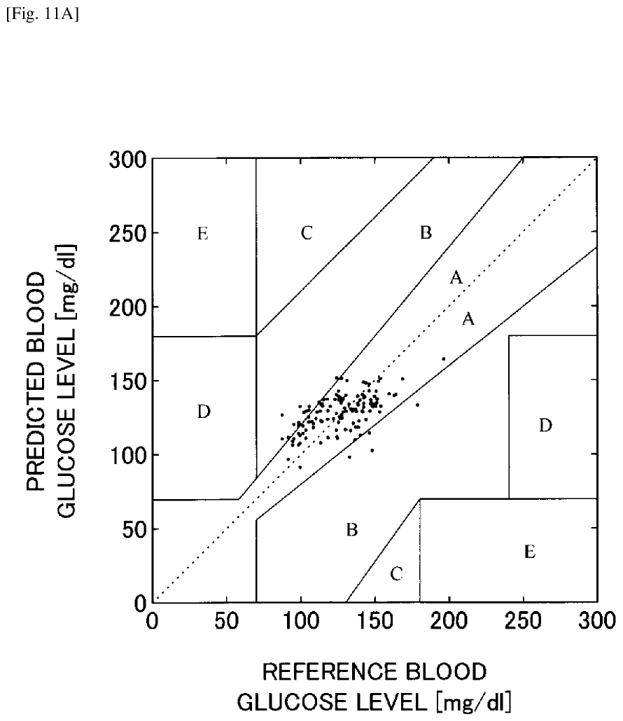

FIG. 10 shows changes in the correlation coefficient with respect to the time delay in series cross validation when the selected wavenumbers are 1050 cm⁻¹, 1070 cm⁻¹, and 1100 cm⁻¹. The correlation is greater than or equal to 0.55 when the time delay is 20 to 30 minutes, and the correlation reaches its maximum value when the time delay is 26 minutes.

For comparison purposes, the dashed line in FIG. 10 indicates changes in the correlation coefficient with respect to the time delay when the selected wavenumbers are 1036 cm¹, 1080 cm¹, and 1110 cm¹ corresponding to the absorption peaks of glucose. Note that with respect to the selected wavenumber 1036 cm⁻¹, although the absorption peak of glucose is actually 1035 cm⁻¹, 1036 cm⁻¹ is selected for convenience because absorbance data is analyzed every 2 cm⁻¹ (see step S11 of FIG. 4). When using the absorption peak wavenumbers of glucose, the correlation coefficients are lower than the correlation coefficients obtained using the wavenumbers selected in the present embodiment. This may be because the absorption spectra measured in vivo overlap with the absorption spectra of many interfering substances. In view of the existence of various interfering substances, the wavenumbers selected in the present embodiment may be more suitable for in vivo measurement as compared with the case of simply focusing on the absorption of glucose and using the absorption peak wavenumbers of glucose. It can be appreciated that in in vivo measurement, a high correlation cannot be obtained when using the absorption peak wavenumbers of glucose.

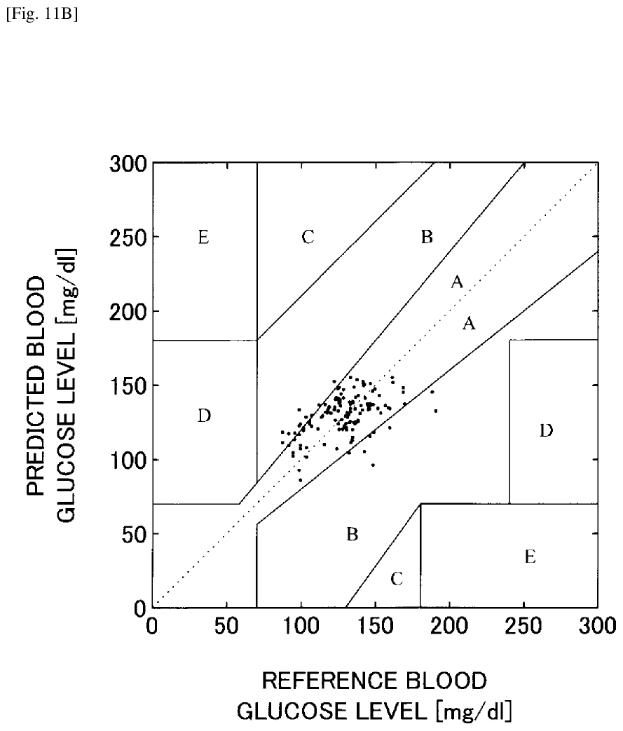

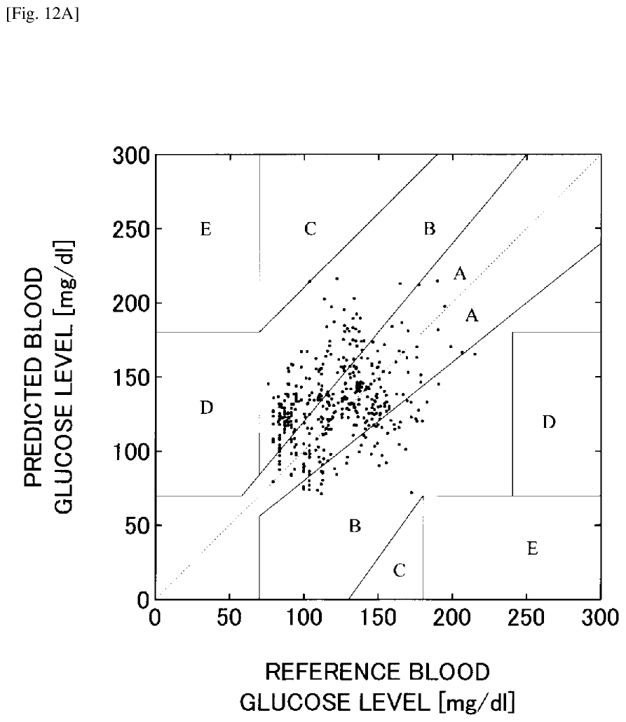

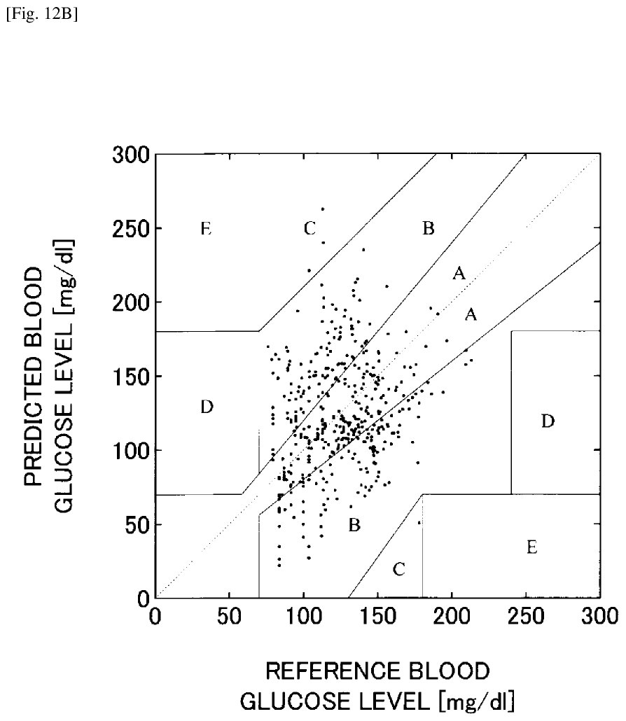

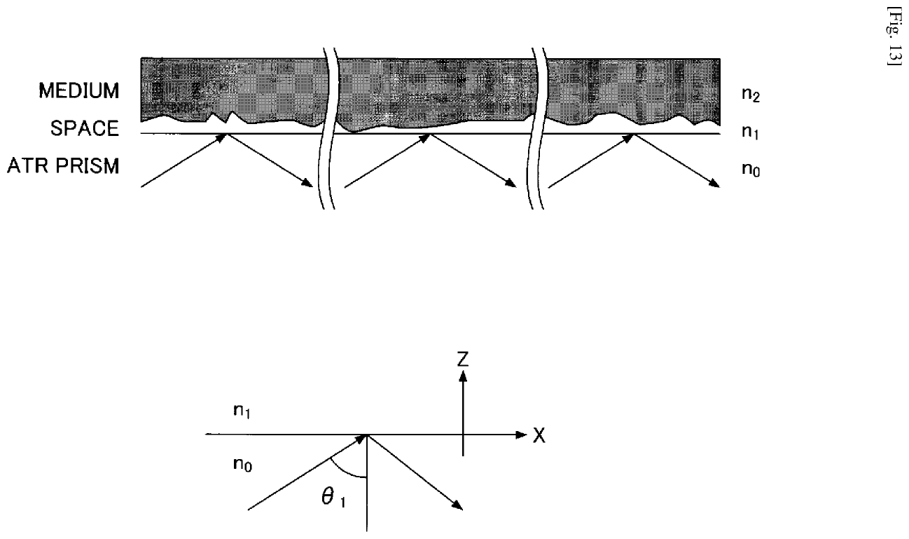

FIGS. 11A-12B represent accuracy evaluation results of step S16 of FIG. 4. FIGS. 11A and 11B represent evaluation results of prediction models based on dataset 1. FIGS. 12A and 12B represent evaluation results of prediction models based on dataset 2. FIG. 11A is a Clarke error grid combining all series of series cross validation for the multiple linear regression model using the wavenumbers 1050 cm^{?1}, 1070 cm^{?1}, and 1100 cm^{?1}. The horizontal axis represents the reference blood glucose level, and the vertical axis represents the predicted blood glucose level. The time delay is set to 26 minutes, which corresponds to the time delay that maximizes the correlation coefficient. Region A contains 86.3% of the samples, which indicates that good accuracy is obtained. That is, the evaluation results indicate the blood glucose level can be accurately measured from the infrared light spectrum using only three wavenumbers.

FIG. 11B is a Clarke error grid combining all series of series cross validation for the PLS regression model that uses a larger number of wavenumbers as a comparison. It is assumed six components with the highest correlation coefficient are used and the time delay is 20 minutes in the PLS regression model. As in the case of using three wavenumbers in the multiple linear regression model, region A contains 86.3% of the samples.

As can be appreciated from FIGS. 11A and 11B that, the Clarke error grids also indicate that the multiple linear regression method using three wavenumbers according to the present embodiment can achieve measurement accuracy comparable to that achieved in the PLS method using a larger number of wavenumbers.

FIG. 12A shows the accuracy evaluation result of dataset 2 predicted using the multiple linear regression model obtained based on dataset 1. In dataset 2, the spectrum data are normalized with respect to the absorbance at 1000 cm^{?1} to eliminate the influence of the difference in the number of reflections between the two prisms used. The prediction model is created using the wavenumbers of 1050 cm^{?1}, 1070 cm^{?1}, and 1100 cm^{?1}, using all the data of dataset 1 normalized to 1000 cm^{?1}, similar to the approach that was followed to process dataset 2. The prediction model obtained can be represented by the following equation (3).

[Math.3]

y=−1175·x(1050 cm⁻¹)+1849·x(1070 cm⁻¹)−859·x(1100 cm⁻¹)+276 (3)

In the above equation (3), y represents the predicted blood glucose level and x(k) represents the measured absorbance at wavenumber k. In FIG. 12A, the correlation coefficient for the three-wavenumber multiple linear regression model is 0.36, and the 100% of the data are within regions A and B.

FIG. 12B is a Clarke error grid for dataset 2 predicted using the prediction model obtained based on dataset 1 using PLS regression as a comparison. The correlation coefficient for the PLS model is 0.25 and 98.8% of the data are within the regions A and B. As can be appreciated from the above, a higher correlation coefficient can be obtained with the three-wavenumber multiple linear regression model according to the present embodiment as compared with the PLS regression model. In the evaluation result of the three-wavelength multiple linear regression model, the p-value for the null hypothesis that there is no correlation is 3.7×10¹⁴, indicating that there is a strong correlation.

Although the conditions of dataset 1 and dataset 2 are different in many respects, correlation can be obtained for dataset 2 without calibration. This indicates that the three-wavenumber multiple linear regression model according to the present embodiment is capable of extracting features suitable for predicting the blood glucose level by regression independent of conditions such as individual differences of subjects and environmental factors. The fact that a higher correlation is obtained for dataset 2 with the three-wavenumber multiple linear regression model as compared with that obtained with the PLS model using a larger number of wavenumbers may be attributed to the improved generalization performance of the estimation model resulting from reducing the number of wavenumbers. Note that accuracy may be further improved by performing calibration with respect to each subject.

The above experimental results demonstrate that appropriate wavenumbers for non-invasive blood glucose measurement are selected in the present embodiment and that the selected wavenumbers and the prediction model have high robustness with respect to blood glucose measurement.

<Optical System Model>

In the following, an optical system model of the ATR prism will be analyzed. The absorption intensity A is measured through the ATR prism. The absorption intensity A is defined by the following equation (4).

$$[ Math .4 ]$$

$$ABSORPTION INTENSITY A = - {log}_{10} ( \frac{I}{{I}_{0}} ) \qquad ( 4 )$$

In the above equation (4), I represents the transmitted light intensity of the ATR prism including the sample and I₀ represents the ATR background noise intensity.

<Reflection in the absence of Space>

First, the influence of light on the medium (e.g., oral mucosa) when there is no space between the ATR prism and the medium will be analyzed. In the following description, it is assumed that n1 represents the refractive index of the ATR prism, and n2 represents the refractive index of the medium. Light incident on the ATR prism is totally reflected on the surface of the medium.

Model dp for single reflection is assumed to represent the penetration depth of an evanescent wave in total reflection. Using the wavelength λ and the refractive indices n1 and n2, the model dp can be represented by the following equation (5).

$$[ Math .5 ]$$

$${d}_{p} = \frac{λ}{2 π \sqrt{{sin}^{2} θ - {( \frac{{n}_{2}}{{n}_{1}} )}^{2}}} \qquad ( 5 )$$

Using the model dp, the absorption intensity A may be represented by the following equation (6).

$$[ Math .6 ]$$

$$\begin{matrix}A = - {log}_{10} ( ATR ) = ( {log}_{10} e ) \frac{{n}_{2}}{{n}_{1}} \frac{{E}_{0}^{2}}{2 cos θ} \frac{{d}_{p}}{2} α \\ = ( {log}_{10} e ) \frac{{n}_{2} {E}_{0}^{2}}{2 cos θ {n}_{1} \sqrt{{sin}^{2} θ - {( \frac{{n}_{2}}{{n}_{1}} )}^{2}}} \frac{{E}_{0}^{2}}{k} α\end{matrix} \qquad ( 6 )$$

Note that the value desired as a measurement value in the above equation (6) is absorption coefficient α per sample film thickness.

A constant term “a” is defined by the following equation (7).

$$[ Math .7 ]$$

$$a = ( {log}_{10} e ) \frac{{n}_{2} {E}_{0}^{2}}{2 cos θ {n}_{1} \sqrt{{sin}^{2} θ - {( \frac{{n}_{2}}{{n}_{1}} )}^{2}}} \qquad ( 7 )$$

The absorption intensity A can be represented by the following equation (8).

$$[ Math .8 ]$$

$$A = \frac{{aE}_{0}^{2}}{k} α \qquad ( 8 )$$

Assuming N represents the number of reflections occurring in the ATR prism, and taking into account the fact that the absorption intensity A is logarithmic, the absorption intensity Aₘ for multiple reflections can be represented by the following equation (9).

$$[ Math .9 ]$$

$${A}_{m} = {\sum }_{n = 1}^{N} \frac{{aE}_{0}^{2}}{k} α \qquad ( 9 )$$

<Reflection in the presence of Space>

Next, reflection in the case where there is a space between the ATR prism and the medium will be contemplated. In practice, space in the form of air space or space formed by liquid such as saliva exists between the ATR prism and the oral mucosa, and the state of the space may change each time a measurement is made to thereby constitute an external disturbance. Accordingly, a multiple reflection model when there is a space between the ATR prism and the medium is contemplated.

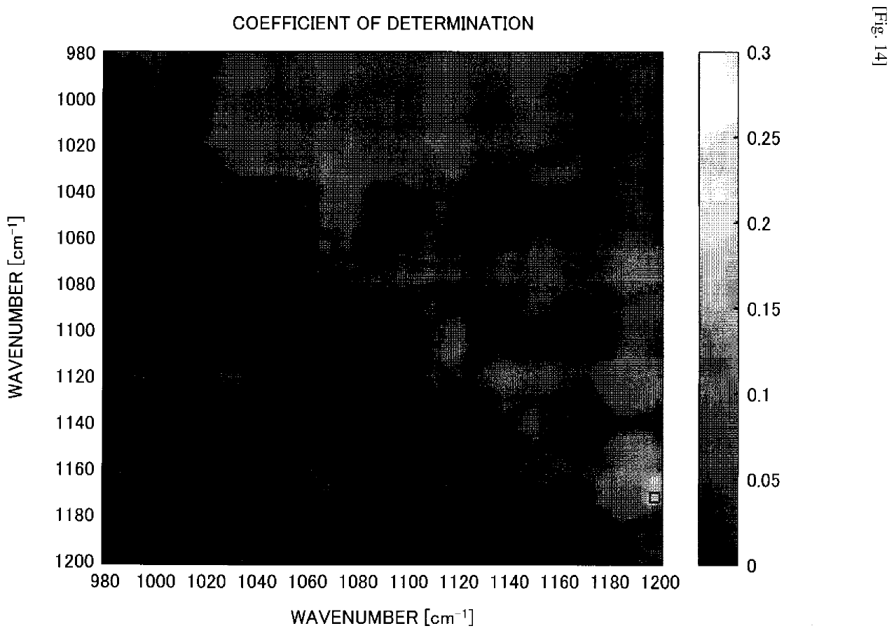

FIG. 13 is a schematic diagram illustrating a case where there is a space between the ATR prism and the measurement surface (e.g., oral mucosa). In the following, it is assumed that n₀ represents the refractive index of the ATR prism, n₁ represents the refractive index of the space, n₂ represents the refractive index of the medium, z represents the space width, and x represents the reflection position. A multiple reflection model in the case where a space exists between the ATR prism and the medium can be represented by the following equation (10).

$$[ Math .10 ]$$

$$E ( x , y ) = {E}_{0} exp ( - i ω t + {ik}_{1} \frac{x}{{n}_{10}} sin {θ}_{1} \} exp ( - {ik}_{2} z \sqrt{{( \frac{sin {θ}_{2}}{{n}_{10}} )}^{2} - 1} ) \qquad ( 10 )$$

An attenuation term “c” is defined by the following equation (11).

$$[ Math .11 ]$$

$$c = exp ( - {ik}_{2} z \sqrt{{( \frac{sin {θ}_{2}}{{n}_{10}} )}^{2} - 1} ) \qquad ( 11 )$$

Based on the above equation (9), taking into account the fact that the attenuation term “c” is negative (c<0), the absorption intensity A_{mz} in the case where there is a space between the ATR prism and the medium can be represented by the following equation (12).

$$[ Math .12 ]$$

$${A}_{mz} = {\sum }_{n = 1}^{N} \{ \frac{a}{k} {E}_{0}^{2} exp ( {ckz}_{n} ) α \} = \frac{{aE}_{0}^{2} α}{k} {\sum }_{n = 1}^{N} \{ exp ( {ckz}_{n} ) \} \qquad ( 12 )$$

Note that because “ckzₙ” can be approximated to zero (0), the Maclaurin series for the term inside “exp” will be as follows.

[Math.13]

exp(x)≈1+x

Thus, the absorption intensity A_{mz} can be represented by the following equation (13).

$$[ Math .13 ]$$

$${A}_{mz} \approx \frac{{aE}_{0}^{2} α}{k} {\sum }_{n = 1}^{N} \{ 1 + {clz}_{n} \} = \frac{{aE}_{0}^{2} α}{k} ( N + ck {\sum }_{n = 1}^{N} {z}_{n} ) \qquad ( 13 )$$

A total value of the space width “zₜ” is defined by the following equation.

$$[ Math .15 ]$$

$${z}_{t} = {\sum }_{n = 1}^{N} {z}_{n}$$

In this case, the absorption intensity A_{mz} can be represented by the following equation (14).

$$[ Math .16 ]$$

$${A}_{mz} = \frac{{aE}_{0}^{2} α}{k} ( N + {ckz}_{t} ) \qquad ( 14 )$$

The influence of the space is in the term (N+ckzₜ), and a measured spectrum is multiplied thereby in the form of a linear equation of wavenumber k.

Note that the value desired as a measurement value is absorption coefficient α per film thickness of the medium. Based on the above equation (14), α can be represented by the following equation (15).

$$[ Math .17 ]$$

$$α = \frac{k}{{aE}_{0}^{2}} \frac{1}{N + {ckz}_{t}} {A}_{mz} \qquad ( 15 )$$

Note the influence of the space is represented by the term (N+ckzₜ) constituting the denominator of the above equation (15).

<Correction of Space Influence>

Assuming the absorption coefficient α in the above equation (15) is constant;

namely, the measurement target is constant, if the variation of the term (N+ckzₜ) can be corrected, the absorption intensity A_{mz} may also be constant. Accordingly, the linear equation (N+ckzₜ) is calculated in the wavelength band at which the absorption coefficient a does not fluctuate, and the measurement of the absorption intensity A_{mz} is divided thereby as indicated by the above equation (15). Also, to cancel the region where the absorption coefficient α does not fluctuate, the absorption intensity A_{mz} is divided by a representative sample spectrum A_{mz}′. Because the representative sample spectrum corresponds to a sample when the total space width zₜ is close to 0 (zₜ?0), a sample with the highest absorbance may be used. Based on the above equation (14), the correction term (N+ckzₜ) may be obtained as follows.

$$[ Math .18 ]$$

$$\frac{{A}_{mz}}{{A}_{mz}^{′}} = \frac{( N + {ckz}_{t} )}{{N}_{ref}} \\ {N}_{ref} \frac{{A}_{mz}}{{A}_{mz}^{′}} = ( N + {ckz}_{t} )$$

Note that N_{ref} is known from the prism design, and as such, the correction term (N+ckzₜ) is obtained by fitting the linear equation to the wave number k.

More simply, if the range of wavenumber k is a small range, k may be regarded as a constant and (N+ckzₜ) may be regarded as a constant independent of the wavenumber k. In this case, a measured absorption spectrum may simply be normalized with respect to a wavenumber at which the absorption coefficient α does not fluctuate, namely, a wavelength exhibiting little absorption of glucose and the like.

<Coefficient of Determination Map for Two-Wavenumber Regression Model>

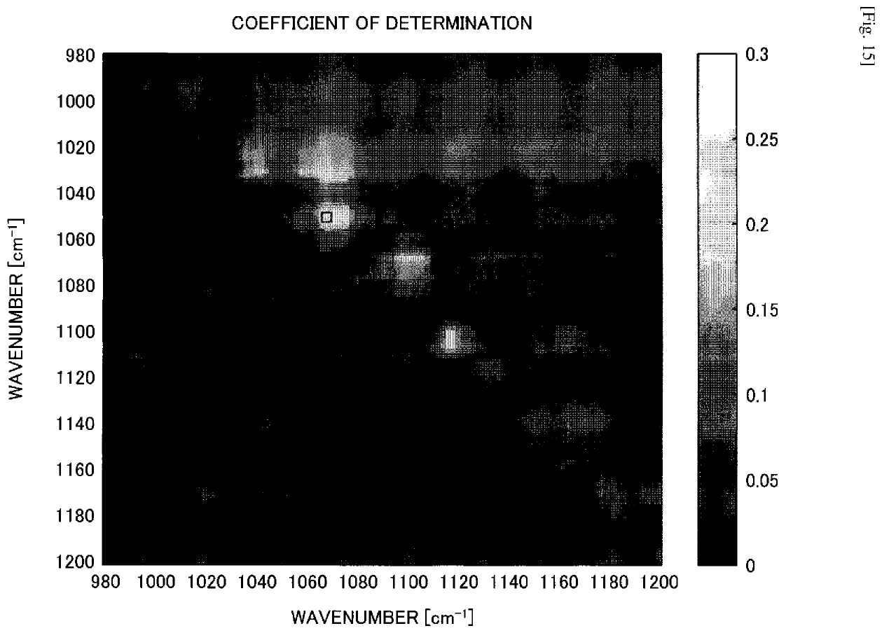

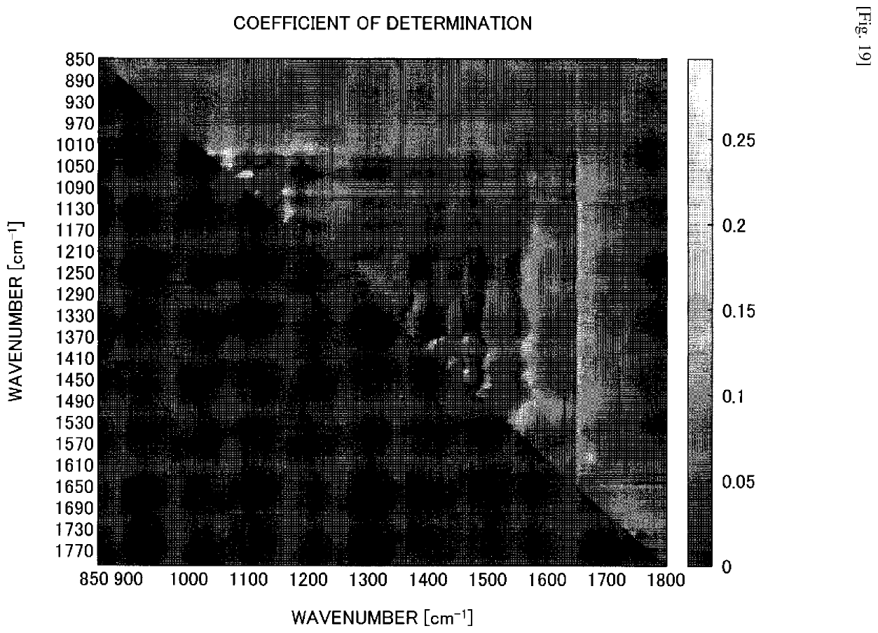

FIGS. 14 to 18 are maps of the coefficient of determination for regression using a multiple linear regression model using two selected wavenumbers (two-wavenumber regression model) where the number of wavenumbers was set to L=2 to select two wavenumbers from a wavenumber range from 980 cm⁻¹ to 1200 cm⁻¹ and the time delay was changed from 0 minutes to 40 minutes. The coefficient of determination (also known as R-squared) is represented by the square of the correlation coefficient and is an index representing prediction accuracy. In the present example, the multiple linear regression model was used to perform regression using all data without cross validation. Note that in the graphs shown in FIGS. 14 to 18, the coefficients of determination are represented in the upper right half, and 0 (zero) is inserted in the lower left half because the results would be the same as the upper right half. Also, note that a region having the maximum coefficient of determination is indicated by a square mark (□) in each of the graphs.

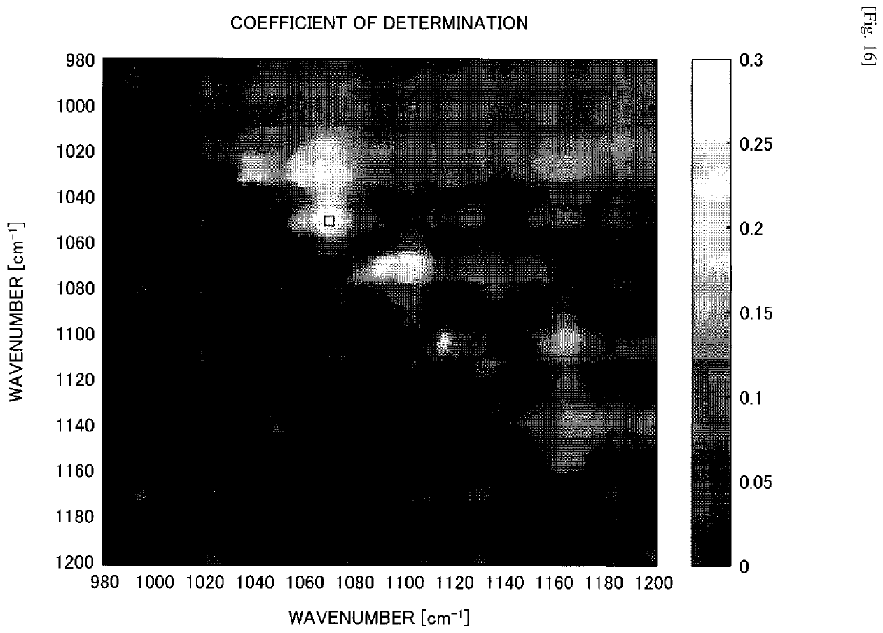

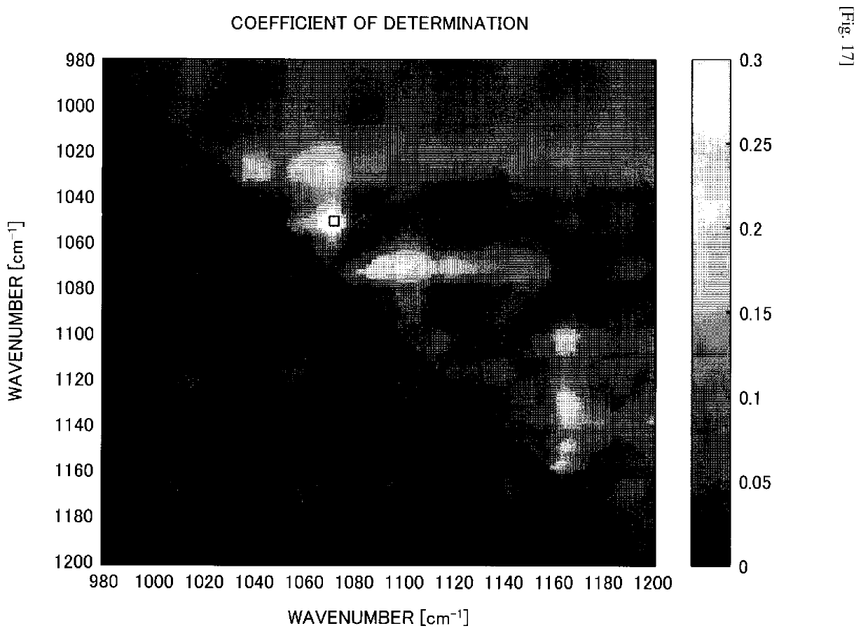

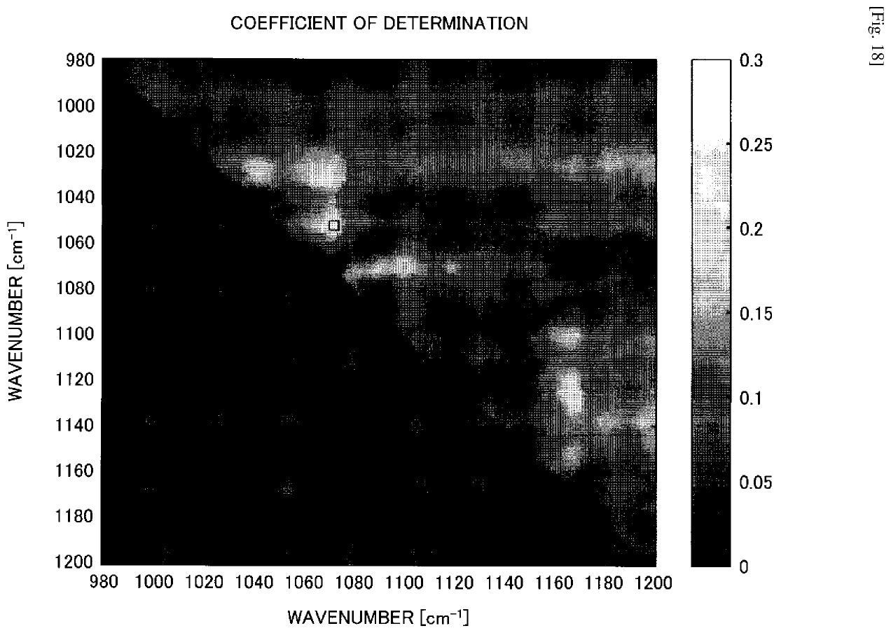

FIG. 14 is a map of the coefficient of determination when the time delay is 0 minutes. As can be appreciated, the map when the time delay is 0 minutes includes a small region with a large coefficient of determination in the vicinity of the wavenumber 1200 cm⁻¹. FIG. 15 is a map of the coefficient of determination when the time delay is 10 minutes. As can be appreciated, the map when the time delay is 10 minutes includes a region with a large coefficient of determination in the vicinity of the wavenumber 1050 cm⁻¹. FIGS. 16 to 18 are maps of the coefficient of determination when the time delay is 20 minutes, 30 minutes, and 40 minutes, respectively. High correlations can be observed when the time delay is 20 minutes (FIG. 16) and when the time delay is 30 minutes (FIG. 17). When the time delay is 20 minutes, the coefficient of determination reaches its maximum value roughly around the wavenumbers 1050 cm⁻¹ and 1070 cm⁻¹. Additionally, peaks are observed around the wavenumbers 1070 cm¹ and 1100 cm¹ and around the wavenumbers 1030 cm⁻¹ and 1070 cm⁻¹. A similar tendency is observed in the map when the time delay is 30 minutes.

FIG. 19 is a map of the coefficient of determination viewed across a wider wavenumber range (850 cm⁻¹ to 1800 cm⁻¹) under the same prediction conditions with a time delay of 20 minutes. Even when the wavenumber range is widened, it can be appreciated when two wavenumbers are selected, high correlation portions are concentrated in the wavenumber range from 980 cm⁻¹ to 1200 cm⁻¹ where the absorption spectrum of glucose exists.

<Wavenumber Combination>

When using a laser as a light source, an increase in the number of wavenumbers used leads to an increase in the number of lasers used. As such, not so many wavenumbers can be selected. That is, the number of wavenumbers to be used is desirably reduced to a small number in order to reduce the size of the measuring device and lower costs. Based on the results described above, the wavenumbers 1050±6 cm⁻¹, 1070±6 cm⁻¹, and 1100±6 cm⁻¹ are desirably selected. Note that spectrum measurement data having a high correlation with the blood glucose level in blood measured by blood sampling corresponds to spectrum measurement data obtained 20 to 30 minutes after measuring the blood glucose level in blood by blood sampling. In other words, the blood glucose level indicated by the infrared spectrum measurement data reflects the blood glucose level in blood from 20 to 30 minutes earlier than the actual spectrum measurement time.

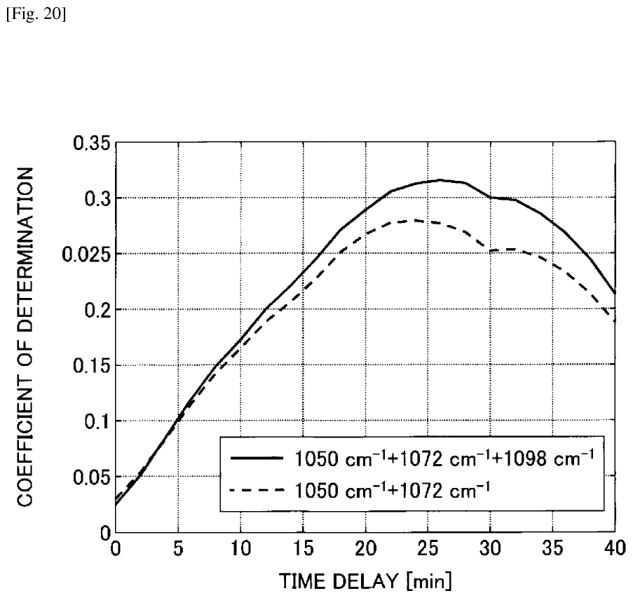

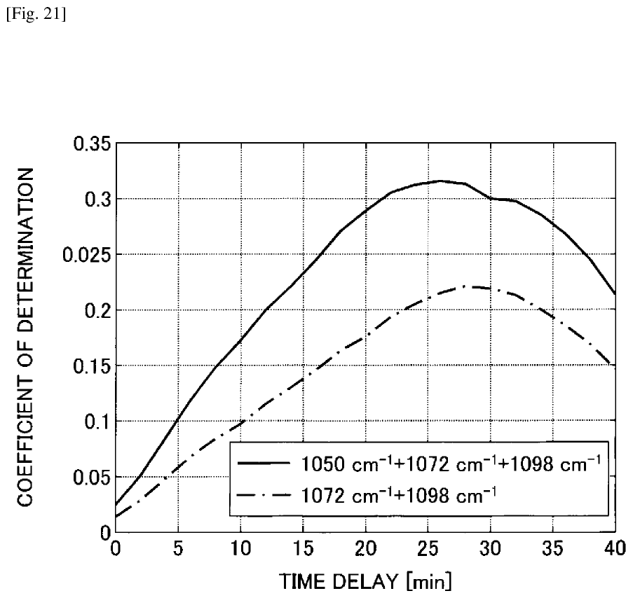

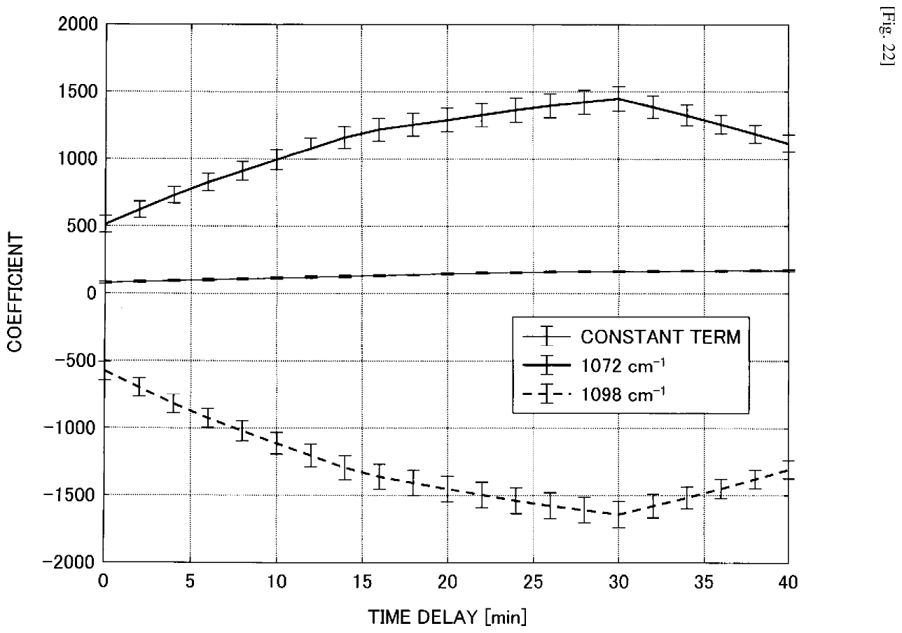

FIGS. 20 and 21 are graphs indicating changes in the coefficient of determination depending on the time delay for differing combinations of candidate wavenumbers obtained by performing coefficient verification by series cross validation. In FIG. 20, the wavenumbers 1050 cm⁻¹, 1072 cm⁻¹, and 1098 cm⁻¹ are selected for a three-wavenumber model, and the wavenumbers 1050 cm⁻¹ and 1072 cm⁻¹ are selected for a two-wavenumber model. In FIG. 21, the wavenumbers 1072 cm⁻¹, 1098 cm⁻¹, and 1050 cm⁻¹ are selected for a three-wavenumber model, and the wavenumbers 1072 cm⁻¹ and 1098 cm⁻¹ are selected for a two-wavenumber model.

With respect to the wavenumber combinations of FIG. 20, the coefficient of determination for the three-wavenumber model is greater than or equal to 0.3 when the time delay is within a range from 20 minutes to 30 minutes, and the coefficient of determination for the two-wavenumber model is greater than or equal to 0.25 when the time delay is within a range from 20 minutes to 30 minutes. With respect to the wavenumber combinations of FIG. 21, the coefficient of determination for the three-wavenumber model is greater than or equal to 0.3 when the time delay is within a range from 20 minutes to 30 minutes as in the case of FIG. 20. The determination coefficient for the two-wavenumber model is the highest when the time delay is within a range from 23 to 33 minutes, but the above time delay range mostly overlaps with the time delay range for the three-wavenumber model.

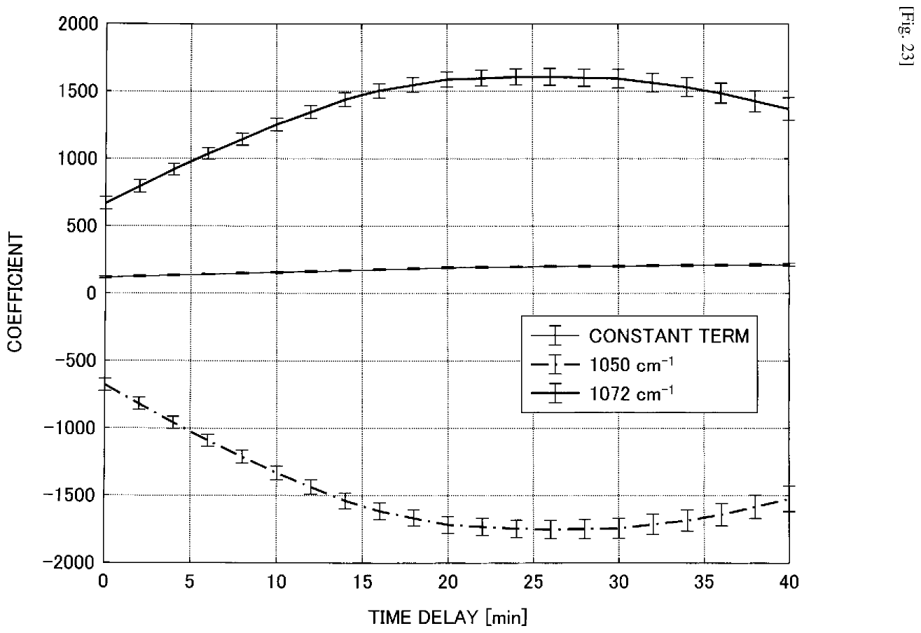

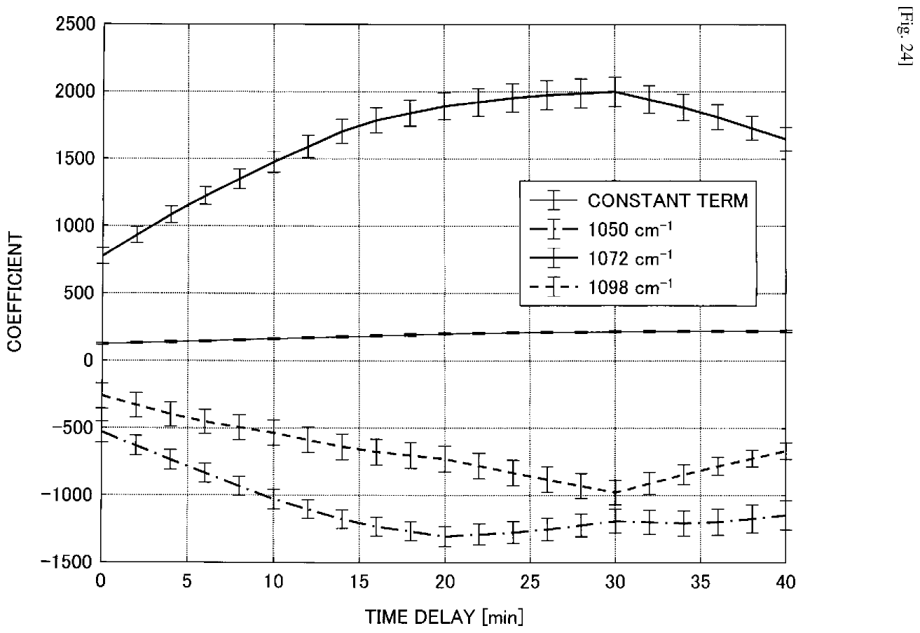

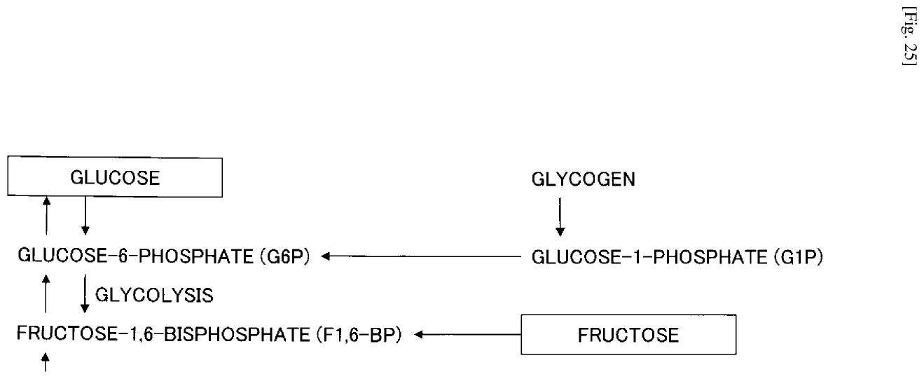

FIGS. 22 to 24 are graphs indicating changes in the regression coefficients as a function of the time delay when certain wavenumbers are selected from candidate wavenumbers. The regression coefficient is the coefficient of each term of the prediction model as represented by the above equation (3). The regression coefficient by which each wavenumber is multiplied changes depending on the time delay. The constant term is constant. In FIG. 22, the wavenumbers 1072 cm⁻¹ and 1098 cm⁻¹ are used. In FIG. 23, the wavenumbers 1050 cm¹ and 1072 cm¹ are used. In FIG. 24, three wavenumbers including 1050 cm⁻¹, 1072 cm⁻¹, and 1098 cm⁻¹ are used. In FIGS. 22 to 24, the regression coefficient of 1072 cm⁻¹ changes in the positive value range, and the regression coefficients of 1050 cm⁻¹ and 1098 cm⁻¹ change in the negative value range as indicated by the prediction model of equation (3).

In FIGS. 22 to 24, the values of the regression coefficients are shown together with error bars representing standard deviations for the results of each series when performing series cross validation. As can be appreciated, the standard deviations are substantially constant even when the time delay changes thereby indicating that the regression coefficients are stably obtained. By using the prediction model according to the present embodiment, highly reliable regression may be implemented.

<In Vivo Glucose Measurement>

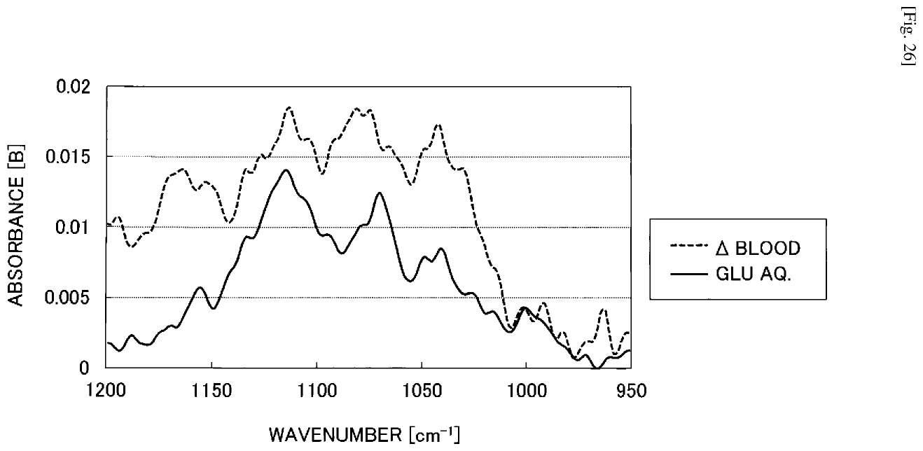

FIG. 25 is a schematic diagram illustrating a part of the glycolysis pathway. Glucose-6-phosphate (G6P) and fructose-6-phosphate (F6P) are the earliest intermediate metabolites of the glycolysis pathway. Glucose-1-phosphate (G1P) is a degradation substance from glycogen stored in cells. As described below, these substances also have absorption spectra in the same wavenumber region as the absorption spectrum of glucose, and it is highly likely that the presence of these substances influence the absorption spectrum being measured.

Because glucose metabolism is involved inside the living body, in vivo glucose measurement is difficult as compared with measuring glucose in a glucose aqueous solution or whole blood. Because the absorption spectrum of a glucose aqueous solution has no interfering substance, the glucose level may be easily measured at the absorption peak wavenumber of glucose. In the case of whole blood, the spectrum may show absorption of other substances, but the substances themselves do not undergo much change and blood glucose level measurement is possible.

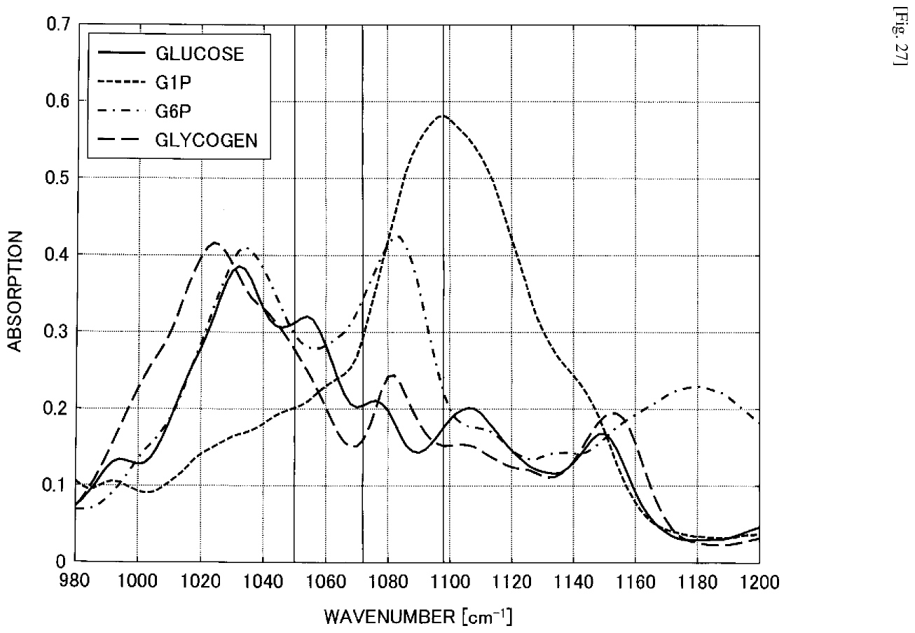

FIG. 26 shows the infrared ATR absorption spectrum of the glucose aqueous solution (denoted as “GLU AQ.”) and the absorption difference spectrum of whole blood samples before and after a meal (denoted as “ΔBLOOD”). In the absorption difference spectrum of whole blood, absorption similar to glucose absorption can be observed in the 900 cm⁻¹ to 1200 cm⁻¹ wavenumber region.

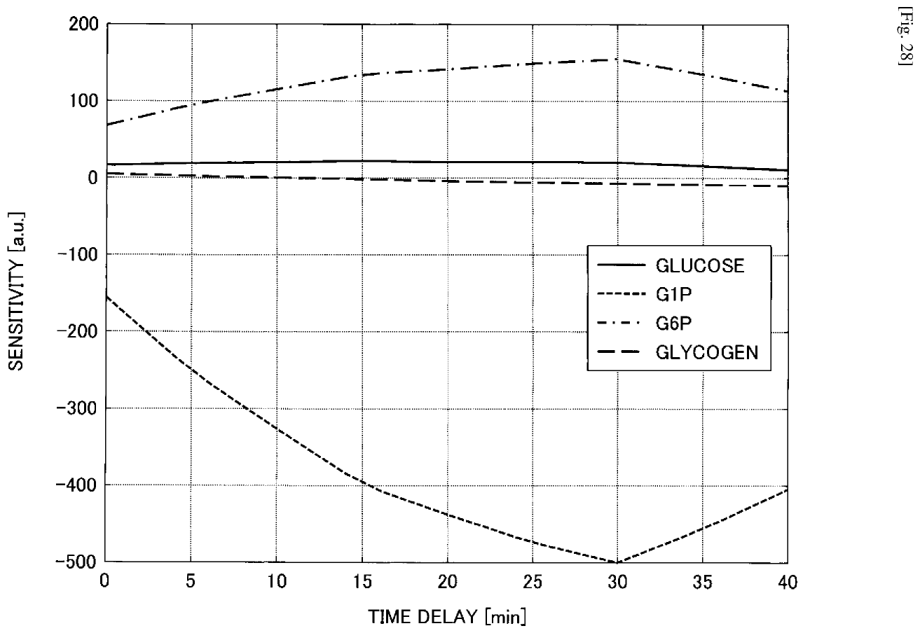

FIG. 27 shows the absorption spectrum of glucose at 10 wt % together with the absorption spectra of metabolite substances (G1P, G6P, and glycogen). Note that in FIG. 27, the wavenumbers 1050 cm⁻¹, 1072 cm⁻¹, 1098 cm⁻¹ selected in the present embodiment are indicated by vertical lines. Of the three wavelengths, 1098 cm⁻¹ corresponds to the peak wavelength of G1P, but the other two selected wavelengths do not overlap with any peaks of the metabolite substances.

In the wavenumber range between one absorption peak and another absorption peak of glucose, such as the wavenumber range between 1035 cm¹ and 1110 cm¹, or the wavenumber range between 1080 cm⁻¹ and 1110 cm⁻¹, the differences between the absorption spectra of glucose and the other metabolite substances are prominently exhibited. Thus, by using the wavenumber range between one absorption peak and another absorption peak of glucose, only the absorption spectrum of glucose can be separated and extracted.

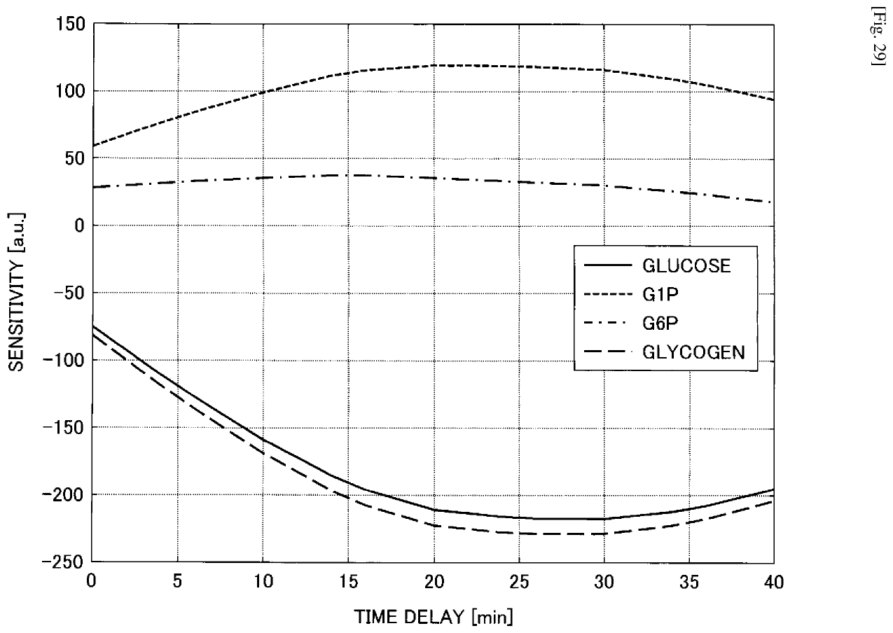

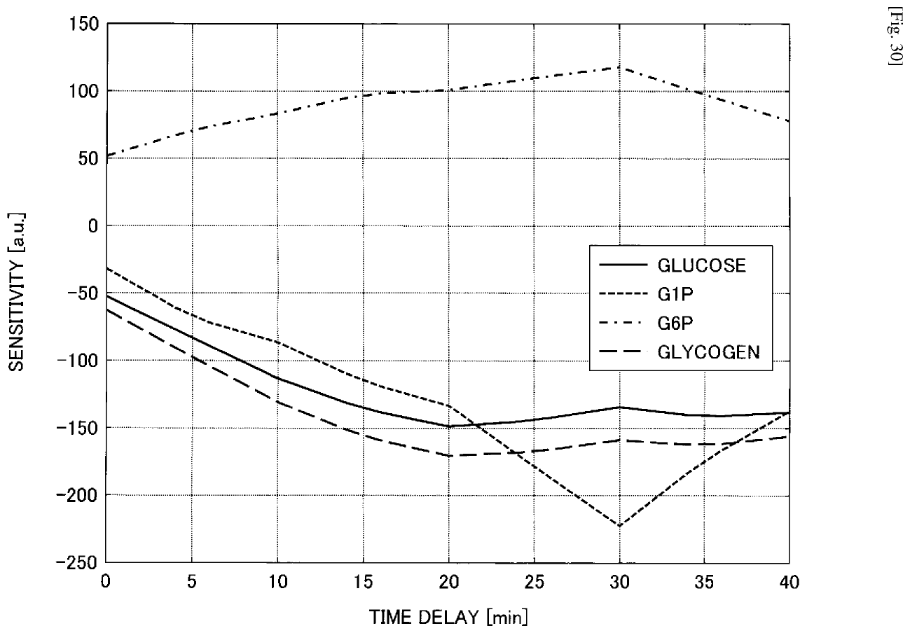

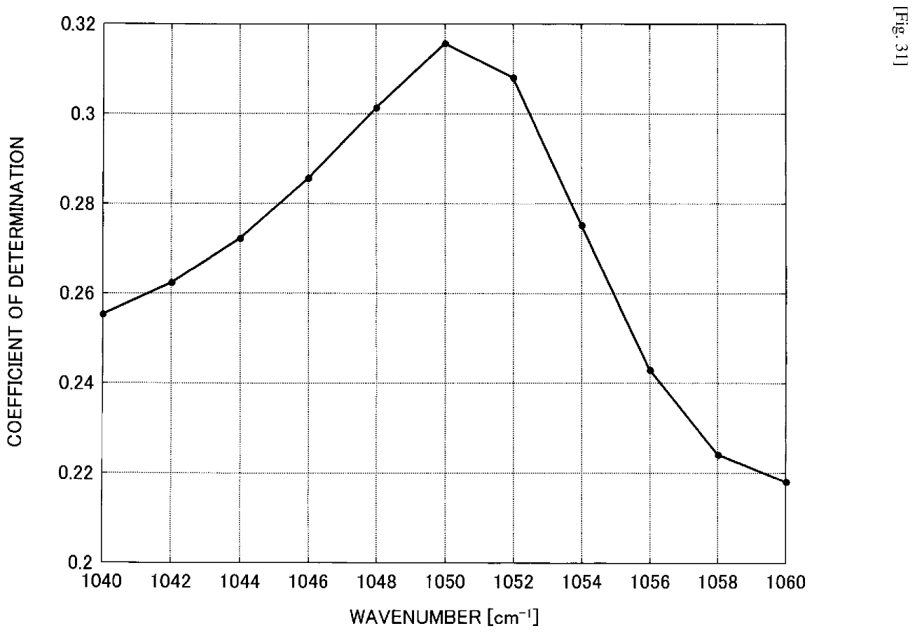

FIGS. 28 to 30 are diagrams showing the sensitivity to each substance when certain wavenumbers are selected. Note that the sensitivity is obtained from the regression coefficients of the prediction model of equation (3) and the absorption spectrum of each substance. FIG. 28 shows the sensitivity in the case of selecting the wavenumbers 1072 cm⁻¹ and 1098 cm⁻¹. FIG. 29 shows the sensitivity in the case of selecting the wavenumbers 1050 cm⁻¹ and 1072 cm⁻¹. FIG. 30 shows the sensitivity in the case of selecting the wavenumbers 1050 cm⁻¹, 1072 cm⁻¹, and 1098 cm⁻¹.

In FIG. 28, the regression coefficients of the two wavenumbers are both negative, and as such, the sensitivity of glucose is indicated as a positive value. In FIGS. 29 and 30, a negative regression coefficient and a positive regression coefficient are included, and as such, the sensitivity of glucose is indicated as a negative value.

The wavenumber 1098 cm⁻¹ used in FIGS. 28 and 30 corresponds to the peak wavelength of G1P, and there is a high possibility that G1P is somehow related to the infrared light measurement spectrum. Further, sensitivity to G6P is also high in FIGS. 28 and 30, and as such, G6P may also be detected.

<Selected Wavenumber Tolerance Evaluation>

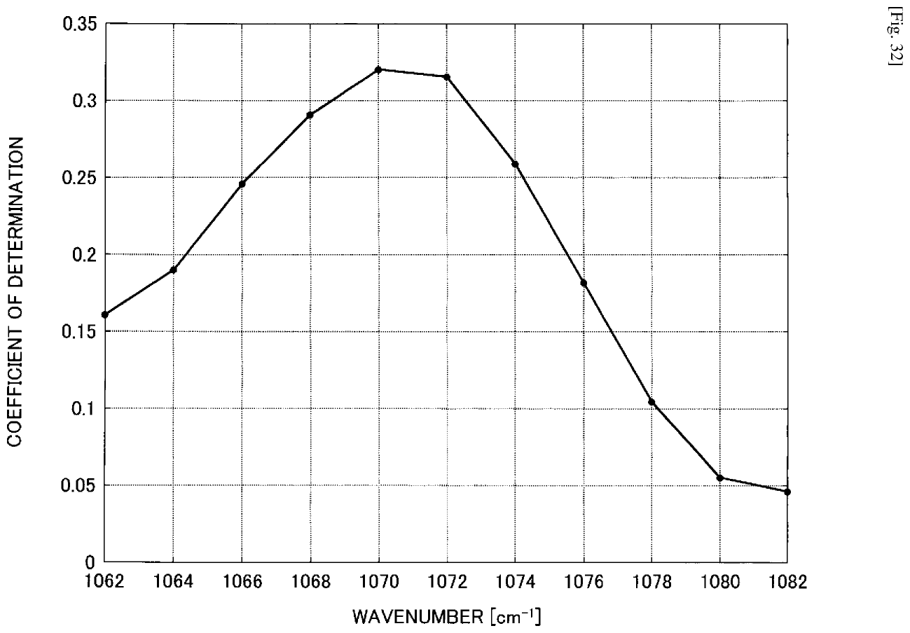

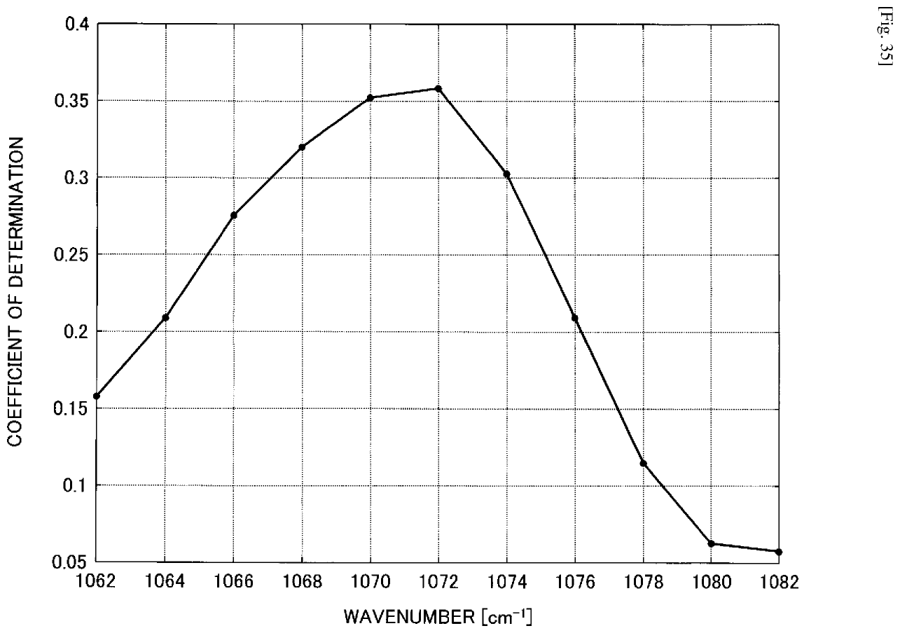

FIGS. 31 to 36 are diagrams showing tolerance evaluations of the selected wavenumbers. FIGS. 31 to 33 show tolerance evaluations when the regression coefficients of the prediction model (e.g., see equation (3)) are adjusted every time the wavenumber is shifted. FIGS. 34 to 36 show tolerance evaluations when the regression coefficients of the prediction model are fixed. The time delay is set to 26 minutes corresponding to when the coefficient of determination is optimized, and evaluations are performed by determining the coefficient of determination when one wavenumber is shifted while the remaining two wavenumbers are fixed. The wavenumber is shifted in increments of 2 cm⁻¹ within a range of ±10 cm⁻¹.

FIGS. 31 to 33 show the extent to which the coefficient of determination decreases in response to a given amount of wavenumber shift when cross series validation is applied; namely, when the regression coefficient of the prediction model is adjusted every time the wavenumber is shifted. With respect to FIG. 31 indicating the coefficient of determination for the 1050 cm¹ band, the coefficient of determination may be greater than or equal to 0.25 by setting the wavenumber to 1050±6 cm⁻¹, and the coefficient of determination may be greater than or equal to 0.3 by setting the wavenumber to 1050±2 cm¹.

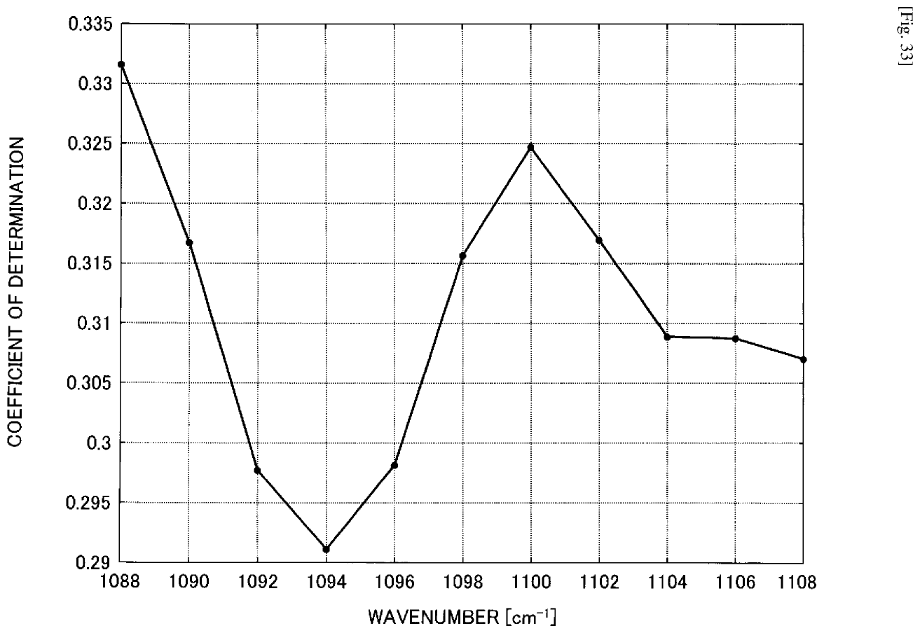

With respect to FIG. 32 indicating the coefficient of determination for the 1070 cm⁻¹ band, the coefficient of determination may be greater than or equal to 0.2 by setting the wavenumber to 1070±6 cm⁻¹, and the coefficient of determination may be greater than or equal to 0.25 by setting the wavenumber to 1070±4 cm⁻¹. Further, the coefficient of determination may be greater than or equal to 0.3 by setting the wavenumber to 1071±2 cm⁻¹.

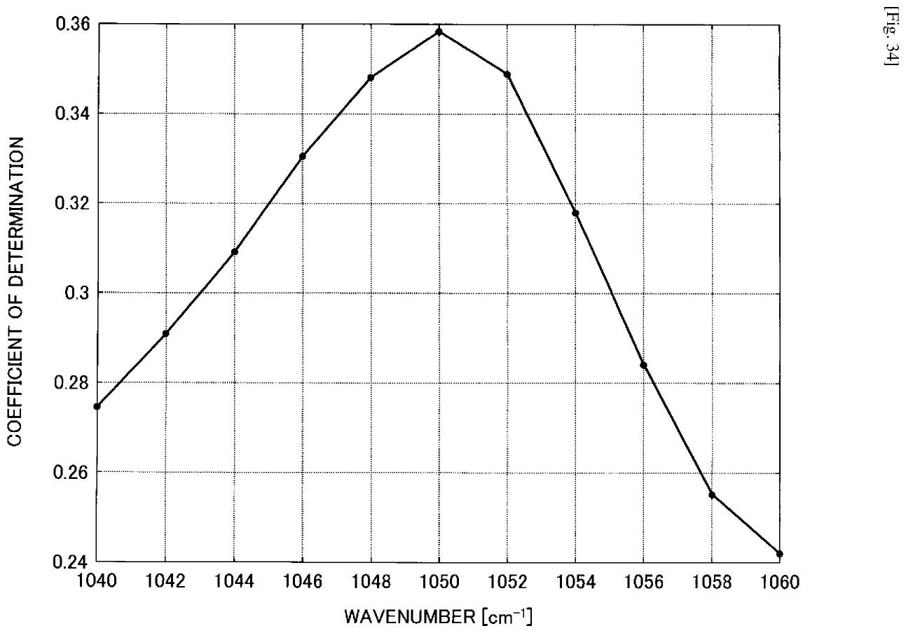

With respect to FIG. 33 indicating the coefficient of determination for the 1100 cm⁻¹ band, it can be appreciated that the 1100 cm⁻¹ band has greater tolerance as compared with the other two wavenumbers. Specifically, the coefficient of determination may be greater than or equal to 0.3 when the wavenumber is in the range of 1100±4 cm⁻¹, and the coefficient of determination may be maintained at 0.29 or higher even when the wavenumber is in the range of 1100±6 cm⁻¹. Note that in FIG. 33, the coefficient of determination is not optimized at the wavenumber 1098 cm⁻¹. This may be attributed to a slight discrepancy between the optimal wavenumber for the data of FIG. 33 and the wavenumber derived from the mode value of the selected wavenumber spectrum as the result of series cross validation. However, an error of 2 cm⁻¹ is an acceptable range that does not substantially affect the variation in the coefficient of determination.

Based on the above results and in view of the configuration of the measuring apparatus, the tolerance range for each selected wavenumber is preferably set to ±6 cm⁻¹. Also, measurement accuracy may be further improved by setting the tolerance range to ±4 cm⁻¹ or ±2 cm⁻¹ as appropriate.

FIGS. 34 to 36 show tolerance evaluations for the same selected wavenumbers as those of FIGS. 31 to 33 when the regression coefficients of the prediction model is fixed. The regression coefficient may be set to the average value of each fold of series cross validation, for example. In the present evaluation, the following equation is used as the prediction model (regression equation).

y=−1160·x(1050 cm⁻¹)+1970·x(1072 cm⁻¹)−978·x(1098 cm⁻¹)+218 [Math.19]

According to the above equation, the regression coefficient of 1050 cm⁻¹ is −1160, the regression coefficient of 1072 cm⁻¹ is 1970, and the regression coefficient of 1098 cm⁻¹ is −978. With the regression coefficients fixed to the above values, one wavenumber is shifted and the coefficient of determination is evaluated.

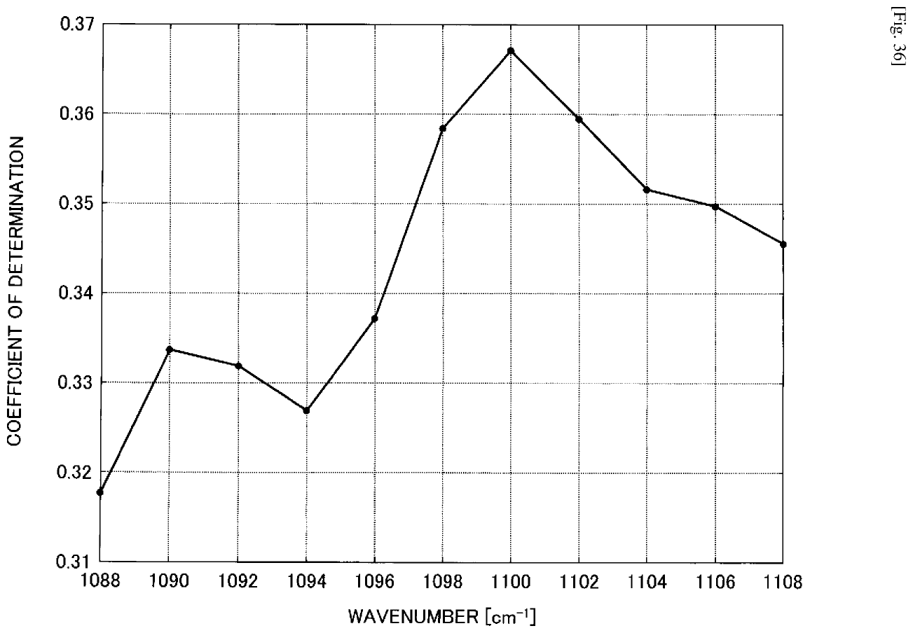

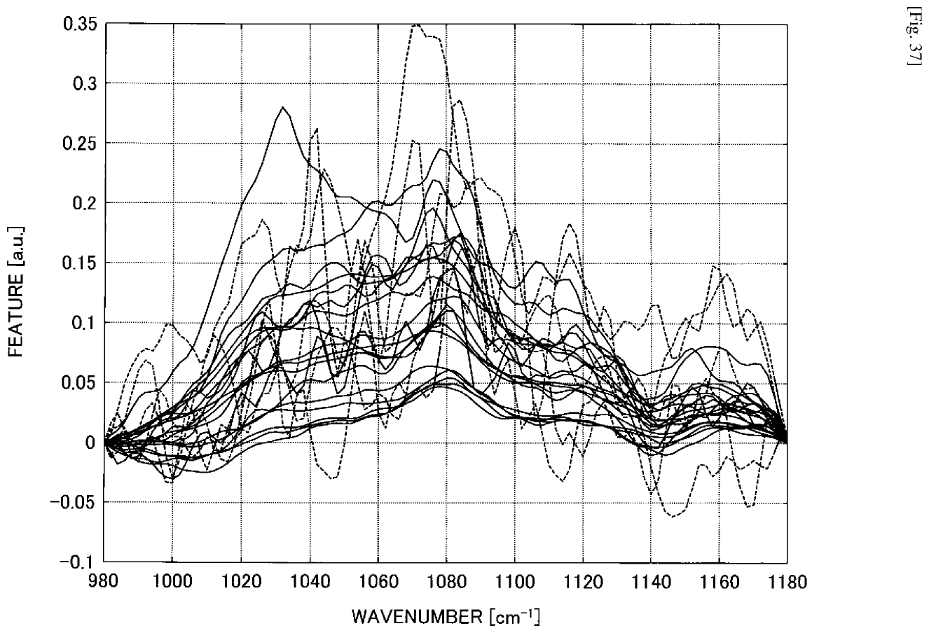

With respect to FIG. 34 indicating the coefficient of determination for the 1050 cm⁻¹ band, the wavenumber deviation (tolerance range) is preferably confined to ±4 cm⁻¹ in order to maintain the coefficient of determination for the 1050 cm⁻¹ band greater than or equal to 0.3. With respect to FIG. 35 indicating the coefficient of determination for the 1070 cm⁻¹ band, the wavenumber deviation is preferably confined to ±2 cm⁻¹ in order to maintain the coefficient of determination for the 1070 cm⁻¹ band greater than or equal to 0.3. With respect to FIG. 36 indicating the coefficient of determination for the 1100 cm⁻¹ band, the wavenumber deviation is preferably confined to ±2 cm⁻¹ in order to maintain the coefficient of determination for the 1100 cm⁻¹ band greater than or equal to 0.35.

<Reliability Output>

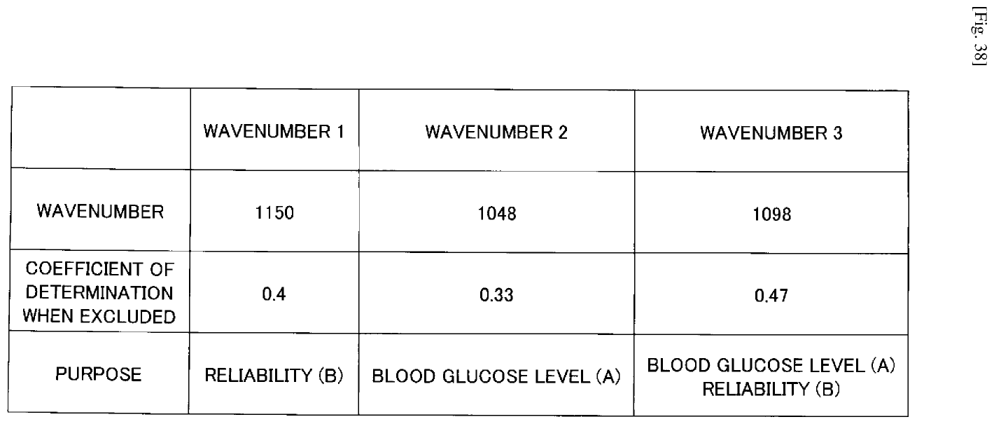

FIG. 37 is a graph illustrating abnormality detection of blood glucose level measurement. Abnormality detection is used when the reliability estimating device 252 of the information processing apparatus 25 outputs the reliability of measurement. When outputting the reliability, the reliability estimating device 252 calculates the LOF (Local Outlier Factor) based on the reconstruction error amount of stacked autoencoders (SAE) of a multilayer neural network, for example. The graph of FIG. 37 shows the LOF output when using two wavenumbers including 1150 cm⁻¹ and 1048 cm⁻¹ for measurement. Note that although 1048 cm⁻¹ corresponds to a blood glucose level measuring wavenumber used in the present embodiment, 1150 cm¹ does not correspond to any of the blood glucose level measuring wavenumbers used in the present embodiment.

In FIG. 37, solid lines represent normal spectrum data and broken lines represent abnormal data. The normal spectrum data have similar spectral shapes and are concentrated in certain regions. The abnormal data have feature values that substantially deviate up and down. The abnormal spectra are clearly distinguished from normal spectra and can be separated. By using a wavenumber other than the blood glucose level measuring wavenumbers for reliability calculation, spectral abnormality can be accurately detected and the accuracy of the reliability output can be improved. By calculating the reliability, when measurement failure occurs due to inadequate contact between the measurement sample and the prism, for example, appropriate measures such as redoing the measurement may be called for to thereby improve measurement accuracy.

Note that in having the reliability estimating device 252 determine whether measurement data corresponds to abnormal data, normal data for each subject may be defined and used for learning, for example. In this way, the reliability may be calculated and output in view of individual differences.

Also, in the case of using a wavenumber other than the blood glucose level measuring wavenumbers for reliability calculation, the number of laser light sources used in the measuring apparatus may have to be increased. In view of the above, for example, two wavenumbers out of three wavenumbers may be used as the blood glucose level measuring wavenumbers, and one wavenumber may be used as a wavelength for reliability calculation. Alternatively, one of two wavenumbers may be used as the blood glucose level measuring wavenumber and the other one of the two wavenumbers may be used as the wavenumber for reliability calculation, for example.

Based on logistic regression analysis, the wavenumbers 1098 cm⁻¹ and 1150 cm⁻¹ may be selected as two wavenumbers that are most suitable for distinguishing abnormal data from normal data. In this case, the accuracy of distinguishing between abnormal data and normal data is 81.8%. Although the wavenumber 1098 cm⁻¹ can be used as a blood glucose level measuring wavenumber, it can also be used as a wavenumber for reliability calculation. For example, at least one of the wavenumbers 1048 cm⁻¹ and 1072 cm⁻¹ may be used for blood glucose level measurement, and the wavenumber 1098 cm⁻¹ may be used for reliability calculation. The wavenumber 1150 cm⁻¹ can be used exclusively as a wavenumber for reliability calculation. Note that when another combination of wavenumbers, 1048 cm¹ and 1150 cm¹, for example, is used for abnormality detection, the accuracy of distinguishing between abnormal data and normal data is 77.2%.

As described above, even when the number of wavenumbers is reduced, by calculating the reliability using a wavenumber different from the wavenumbers used for blood glucose level measurement, the accuracy of the reliability output by the reliability estimating device 252 can be improved.

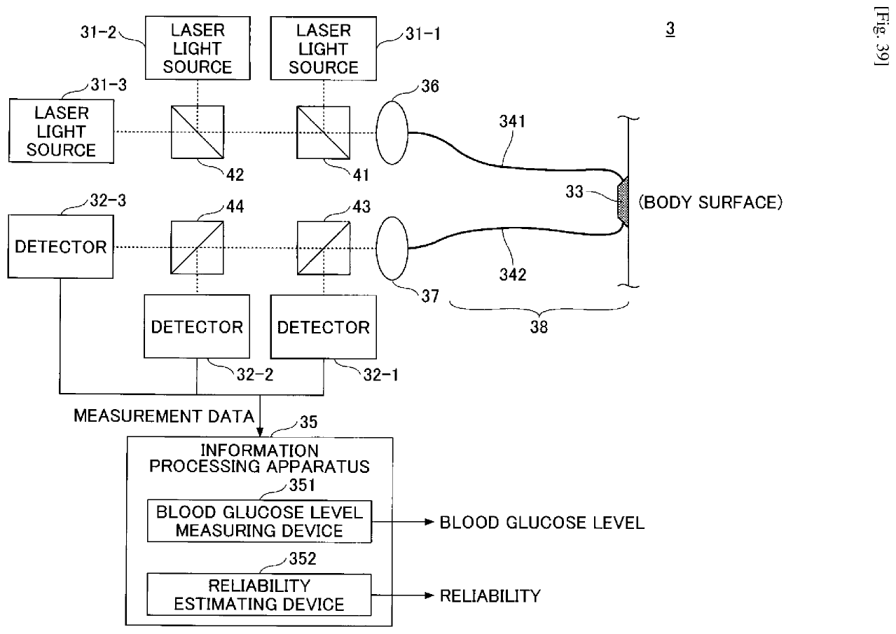

FIG. 38 is a table indicating the coefficient of determination for blood glucose level regression when one wavenumber out of three wavenumbers to be used is excluded. In the present example, 1150 cm¹ as wavenumber 1, 1048 cm¹ as wavenumber 2, and 1098 cm⁻¹ as wavenumber 3 are used. When wavenumber 1 is excluded, the coefficient of determination is 0.4. When wavenumber 2 is excluded, the coefficient of determination is 0.33. When wavenumber 3 is excluded, the coefficient of determination is 0.47. As can be appreciated, a relatively high coefficient of determination can be maintained even when wavenumber 1 or wavenumber 3 is excluded from blood glucose measurement. Thus, even when these wavenumbers are used for reliability calculation (excluded from blood glucose measurement), the impact of the exclusion on the coefficient of determination representing the blood glucose level prediction accuracy may be relatively small. On the other hand, when wavenumber 2 is excluded, the coefficient of determination decreases to 0.33, indicating that the correlation is weakened.

As can be appreciated from the above, wavenumber 1 is to be used exclusively for reliability calculation, wavenumber 2 is to be used exclusively for blood glucose level measurement, and wavenumber 3 can be used for both reliability calculation and blood glucose level measurement.

The results indicated in FIG. 38 may be expressed as follows.

When predicting (regressing) the blood glucose level by combining a data group of blood glucose level measuring wavenumbers and a data group of wavenumbers for reliability estimation, assuming A denotes the prediction accuracy when excluding data relating to one wavenumber included in the data group of the blood glucose level measuring wavenumbers, and B denotes the prediction accuracy when excluding data relating to one wavenumber included in the data group of wavenumbers for reliability estimation, the following relationship holds: (Any Value of B)≥(Maximizing Value of A).

That is, the prediction accuracy when excluding data relating to a wavenumber for reliability estimation is always greater than or equal to the maximum prediction accuracy when excluding data relating a blood glucose measuring wavenumber. Note that the coefficients of determination for regression as indicated in FIG. 38 may be used as the prediction accuracy, for example. According to an aspect of the present embodiment, by using three wavenumbers, both the blood glucose level and the reliability (normal data/abnormal data determination) can be accurately output.

### MODIFICATION EXAMPLE

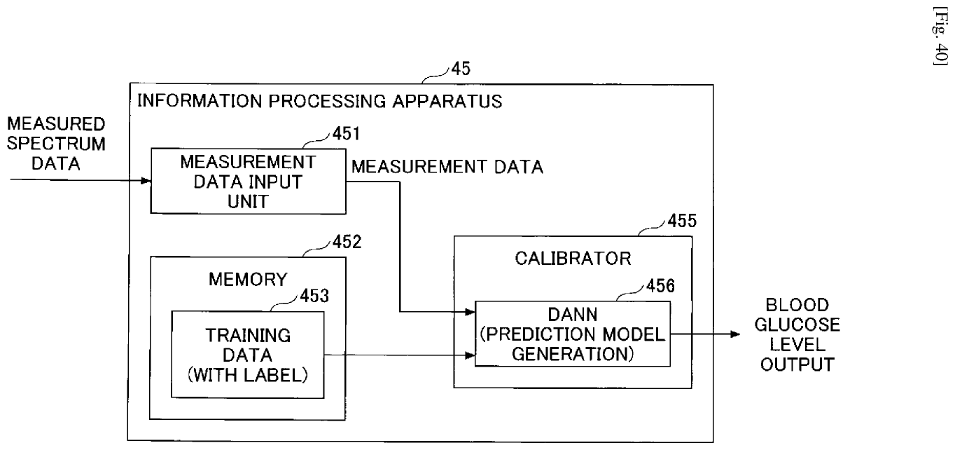

FIG. 39 is a schematic diagram illustrating a configuration of a measuring apparatus 3 according to a modification example. The measuring apparatus 3 includes a first laser light source 31-1, a second laser light source 31-2, a third laser light source 31-3, an ATR prism 33, a first detector 32-1, a second detector 32-2, a third detector 32-3, and an information processing apparatus 35. The measuring apparatus 3 also includes dichroic prisms 41 to 44 and collimator lenses 36 and 37.

Beams in the infrared region that are output from the laser light sources 31-1 to 31-3 are combined into a single optical path by the dichroic prisms 41 and 42, and are condensed on the hollow optical fiber 341 by the collimator lens 36. Infrared light propagated through the hollow optical fiber 341 undergoes attenuation at the ATR prism 33 according to the infrared light absorption spectrum of a sample or a body surface (oral mucosa) in contact with the ATR prism 33. Reflected light carrying blood glucose level information of the sample is incident on the collimator lens 37 from the hollow optical fiber 342. The ATR prism 33 and the hollow optical fibers 341 and 342 constitute an ATR probe 38. The reflected light is condensed by the collimator lens 36 onto the dichroic prism 43, and light of a first wavenumber is detected by the first detector 32-1. Light of a second wavenumber that is included in light transmitted through the dichroic prism 43 is reflected by the dichroic prism 44 and detected by the second detector 32-2. The light transmitted through the dichroic prism 44 is detected by the third detector 32-3. The detection results of the first detector 32-1, the second detector 32-2, and the third detector 32-3 are input to the information processing apparatus 35. A blood glucose level measuring device 351 of the information processing apparatus 35 determines a blood glucose level based on a prediction model using measurement data obtained with blood glucose level measuring wavenumbers and outputs the determined blood glucose level. A reliability estimating device 352 of the information processing apparatus 35 estimates measurement reliability using data obtained with a wavenumber for reliability estimation and outputs the estimated reliability.

Of the three wavenumbers used in the measuring apparatus 3, two wavenumbers corresponding to wavenumbers that are in between absorption peaks of glucose are selected as blood glucose measuring wavenumbers, and one wavenumber that differs from the blood glucose level measuring wavenumbers is used as a wavenumber for reliability estimation. The measuring apparatus 3 can perform measurement free from influences of individual differences between subjects and changes in environmental conditions and can accurately calculate the blood glucose level in the living body where metabolites and other substances are present. The measuring apparatus 3 can also accurately calculate and output the measurement reliability.

Note that embodiments of the present invention are not limited to blood glucose level measurement. The measurement target is not limited to glucose, and technical concepts such as wavenumber (wavelength) selection and determination according to the above-described embodiment of the present invention can also be applied to the measurement of other components in the living body such as proteins, cancer cells, and the like.

The multiplexing element/demultiplexing element used in the modification example of FIG. 39 is not limited to a dichroic prism. For example, a spectroscopic element using a half minors or diffraction may also be used. The light source is not limited to a laser light source; for example, a combination of a light source that emits light of a wide wavelength range and a spectroscope may be used. In the case of using a laser light source, instead of combining multiple laser outputs as describe above, in some embodiments, the light emission time of a plurality of laser light sources may be switched in time series, for example. In this case, the number of laser light sources may be further reduced, and for example, the measuring apparatus may have one detector for receiving light.

The number of the laser light sources in FIG. 39 is not limited to three, and for example, a first laser light source that outputs light of 1048±6 cm⁻¹ and a second laser light source that outputs light of 1098 cm⁻¹ may be used to radiate light of two wavenumbers to determine the blood glucose level. Alternatively, light of 1048 cm⁻¹ may be used for blood glucose measurement and light of 1098 cm⁻¹ may be used for reliability estimation such that the reliability of measurement may be estimated.

Also, note that the wavenumber used for normalizing a dataset for generating a prediction model is not limited to 1000 cm⁻¹ and may be some other wavenumber in the mid-infrared region other than the blood glucose measuring wavenumbers. For example, a wavenumber less than or equal to 1035 cm⁻¹ or a wavenumber greater than or equal to 1110 cm⁻¹ may be used for normalization.

<Calibration Applying DANN>

In the following, calibration will be described. Generally, in noninvasive blood glucose measurement technology, calibration is implemented with respect to each individual or at periodic intervals in order to ensure robustness with respect to various conditions including individual differences or to maximize the correlation between the blood glucose level in blood measured by blood sampling and measurement data obtained by noninvasive blood glucose measurement. In such calibration process, the blood glucose level in blood has to be measured by blood sampling in order to obtain training data. In other words, invasive blood glucose measurement is ultimately required in order to perform accurate measurement. Note that the above-described technique of Patent Document 2 also fails to solve the problem of requiring blood sampling for calibration purposes.

Also, there are individual differences among users who use the measuring apparatus according to the present embodiment, and in order to maximize the correlation between noninvasively obtained measurement data and the actual blood glucose level for each user, calibration is preferably performed automatically at the user site. Conventionally, blood sampling has been required to measure the blood glucose level in the blood of the user and use the measurement as training data. However, in the present embodiment, calibration is performed using measured spectrum data rather than using the blood glucose level in the blood of the user as training data.

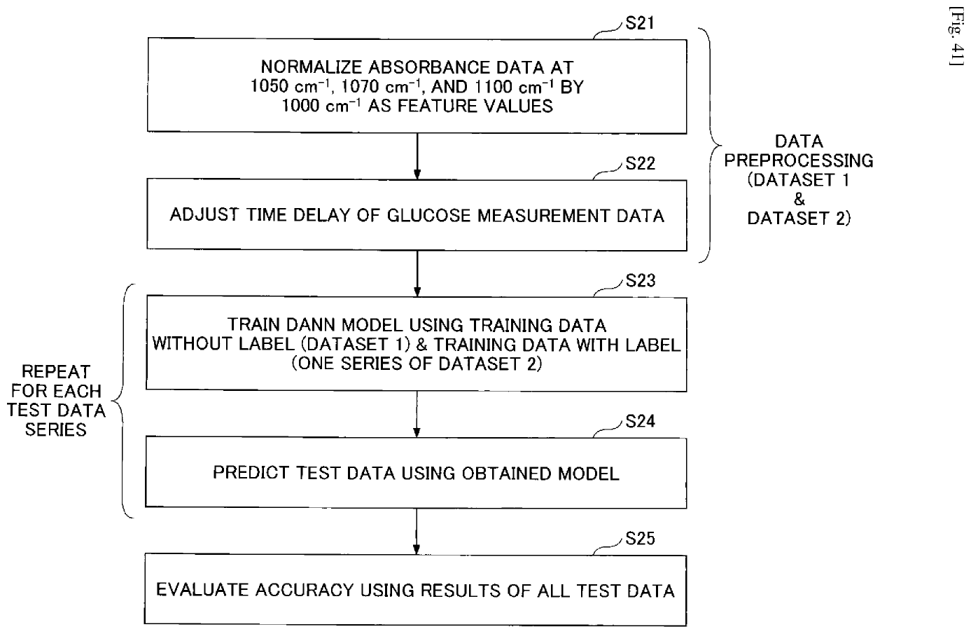

FIG. 40 is a block diagram illustrating a functional configuration of an information processing apparatus 45 that performs noninvasive calibration in the measuring apparatus according to the present embodiment. The information processing apparatus 45 includes a measurement data input unit 451 that inputs measured spectrum data obtained using mid-infrared light, a memory 452 that stores training data 453 collected in advance, and a calibrator 455 that calibrates the blood glucose level measurement using measurement data and training data 453. The calibrator 455 generates a prediction model using DANN (Domain Advisory Neural Network) that performs adversarial learning as a neural network and outputs a blood glucose level based on the prediction model. This prediction model has a domain adaptation (DA) function.

The measurement data is spectrum data optically measured at the mucous membrane such as the inner lip using a specific wavenumber (or wavelength) selected from the mid-infrared region excluding the absorption peaks of glucose. In the calibration of the measurement data, labeling of blood glucose levels is not required and blood sampling is not required. Because the prediction model for regression (prediction) of the blood glucose level based on spectrum data has a domain adaptation (DA) function, calibration can be performed by learning without labels.

Domain adaptation is a form of transfer learning that involves applying learning results in a certain task to other tasks. When training data (also referred to as “learning data”) and test data for evaluation have different distributions, training data with a teaching label is used to accurately make predictions on test data having a different distribution from the training data.

The calibrator 455 uses the input measured spectrum data as test data for evaluation and also incorporates the measured spectrum data in the training data 453 retrieved from the memory 452 for use as training data.

In the following, evaluation of the processing function of the calibrator 455 according to the present embodiment using the same dataset 1 and dataset 2 illustrated in FIG. 3 will be described.

Dataset 1 is a dataset including data obtained from a single subject on different occasions, and dataset 2 is a dataset including data obtained from five subjects (different from the subject of dataset 1) on a plurality of occasions.

FIG. 41 is a flowchart illustrating a process flow of the calibrator 455 relating to pre-processing, learning, and evaluation of a regression result.

First, the wavenumbers 1050 cm¹, 1070 cm¹, and 1100 cm¹ are used as working wavenumbers for regression of the blood glucose level, the absorbance data at the respective wavenumbers are normalized with respect to the absorbance at 1000 cm⁻¹, and the normalized data are used as feature values (step S21).

Because it takes some time for the glucose level in the interstitial fluid and the intra-cellular metabolic system to reach the glucose level in the blood vessel, the delay time of measurement data is adjusted to reflect the above delay (step S22). In the present embodiment, as described above, measurement data is delayed by 20 to 30 minutes, preferably 26 minutes (i.e., measurement data is regarded as data representing the blood glucose level in blood from 26 minutes earlier). Note that steps S21 and S22 correspond to pre-processing process steps.

The dataset 1 and dataset 2 that have undergone preprocessing are used to train a DANN model. Specifically, dataset 1 is used as training data with a blood glucose level label, and each data series of dataset 2 is used as unlabeled test data to train the DANN model (step S23). Then, the test data is predicted using the obtained model (step S24). Note that steps S23 and S24 correspond to learning process steps. Steps S23 and S24 are repeated until learning of all the data series is completed.

When learning is completed with respect to all the data series, accuracy is evaluated by combining the results of all the test data (step S25). The accuracy evaluation is performed with respect to all data of dataset 2 by implementing series cross validation for each data series. Note that step S23 corresponds to an evaluation process step.

In the learning process of steps S23 and S24, to implement domain adaptation (DA), the data of dataset 2 corresponding to test data are also used as training data without blood glucose level labels.

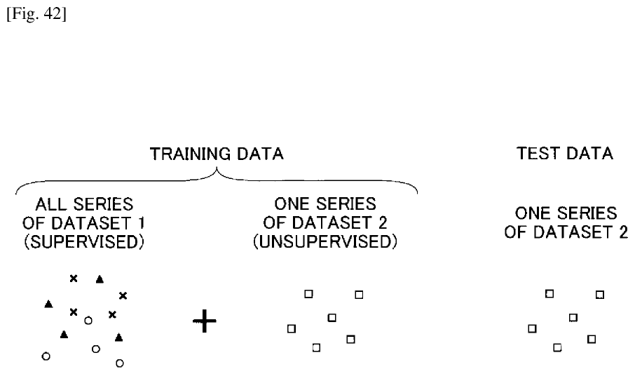

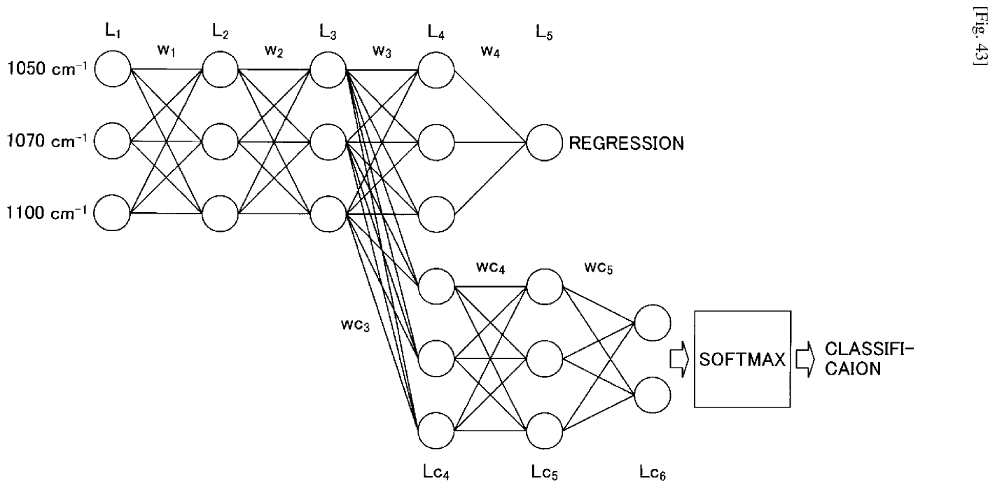

FIG. 42 illustrates handling of training data and test data. The test data for evaluation corresponds to one series of data of dataset 2 (unsupervised data). On the other hand, the training data includes all series of data of dataset 1 (supervised data) and one series of data of dataset 2 (unsupervised data).

Note that the differences in the shapes of the data points in FIG. 42 represent differences in the data series. For training (or learning), all series of data of dataset 1, which includes data with blood glucose level labels, and one series of data of dataset 2, which includes unlabeled data, are used. For evaluation, the same one series of data of dataset 2 used for training is used. The above processes are repeated with respect all series of data of dataset 2 to evaluate prediction accuracy. Note that data of dataset 2 is not labeled with blood glucose level teaching data even when used during training. As such, although the same series of data of dataset 2 is used for training and evaluation, the true value of the blood glucose level is not given at the time of training.

FIG. 43 illustrates a configuration of a network used in the calibrator 455. The absorbance at 1050 cm¹, 1070 cm¹, and 1100 cm¹ are input to the network. The network includes a regression network and a classification network. In FIG. 43, Lₓ denotes each layer of the regression network, and Lₑₓ denotes each layer of the classification network. The regression network branches at layer L₃ to be connected to the classification network. wₓ and wₑₓ respectively denote the weights of the networks at the corresponding layers.

A Leaky Rectified Linear Unit (ReLU) with a gradient of ai=0.2 in the negative region is used as the activation function. Euclidean loss is used as the loss function for regression, and Softmax Cross Entropy is used as the loss function for classification. Also, batch normalization is used for each layer. Adam (adaptive moment estimation) is used as an optimization method.

As described below, because the classification network updates weights w_{c3} to w_{c5} to discriminate or identify dataset 1 and dataset 2, the classification network may also be referred to as a “discriminator”.

The regression network updates learning of the prediction model so that dataset 1 and dataset 2 cannot be distinguished based on the learning result of the classification network (discriminator).

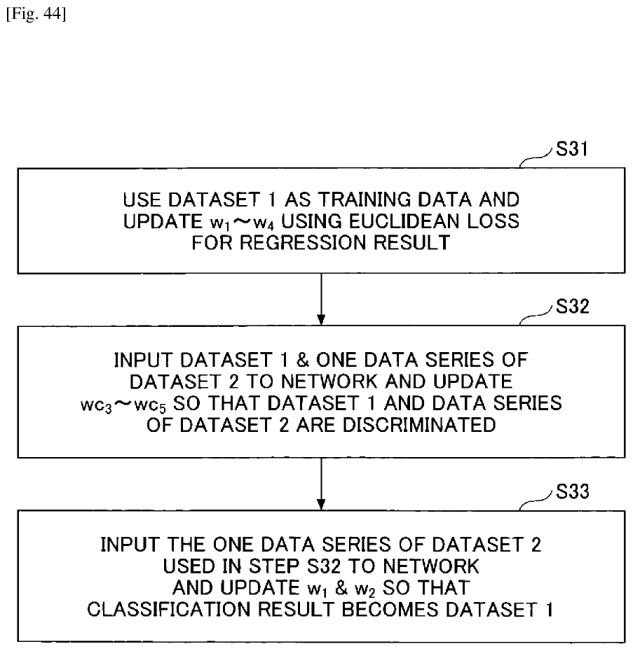

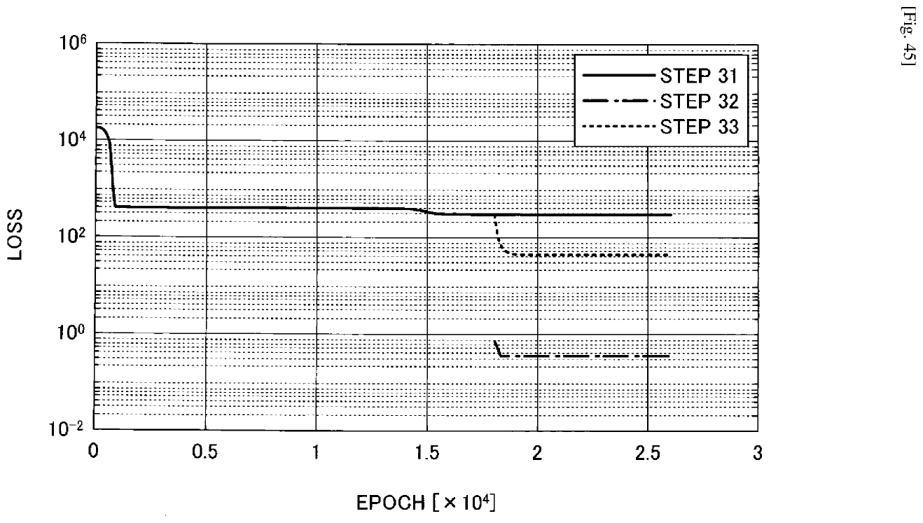

FIG. 44 is a flowchart illustrating a learning process using the network of FIG. 43. By updating the weights in steps S32 and S33 of FIG. 44, regression with high accuracy can be performed while overlapping the distributions of dataset 1 and dataset 2 in layer L₁ to layer L₃.

First, in step S31, the absorbance data of the input dataset 1 is used as training data to train the network for performing regression of the blood glucose level. At this time, weights w₁ to w₄ of layers L₁ to L₄ are updated using Euclidean loss of the regression result.

Then, in step S32, one series of absorbance data without label data of dataset 2 is added as input data in addition to dataset 1 to train the network for distinguishing between data of dataset 1 and data of dataset 2. The training (learning) is performed in the classification network or discriminator. The one series of data of dataset 2 is used as adversarial data. Adversarial data is data that is added as deliberate noise to training data in a small amount to cause output of predictions that are significantly different from that for original training data. A technique for improving the performance of a prediction model by training the network to output a prediction for adversarial data that is similar to the prediction for original training data is referred to as adversarial learning.

At the same time as step S32, in step S33, weights w₁ and w₂ of the regression network are updated so that dataset 1 and dataset 2 cannot be distinguished. In this way, a feature value that enables regression of the blood glucose level and does not enable distinction between dataset 1 and dataset 2 is extracted at the output of layer L₃. As a result, a network for estimating the blood glucose level is trained while correcting the deviation of the distributions of dataset 1 and the one series of data of dataset 2 that has been input.

The learning method and parameters in the process flow of FIG. 44 are as follows. During the first 1800 epochs, learning of the network involves executing only step S31 using supervised data of dataset 1 to learn weights w₁ to w₄.

Thereafter, steps S32 and S33 are executed at the same time in addition to step S31 to promote learning using unsupervised data of dataset 2 in addition to dataset 1. In step S33, in order to balance regression performance and domain adaptation, only an iterative process in which the regression loss value for step S31 is less than 320 is performed, and the loss value for step S33 is multiplied by 350 in order to achieve balance with the losses for steps S31 and S32. A total of 2600 epochs are run before learning is completed.

FIG. 45 is a graph representing changes in the loss for each step of the learning process of the model in a representative series of dataset 2. The solid line represents the loss with respect to step S31 of FIG. 44, the long dashed short dashed line represents the loss with respect to step S32, and the dotted line represents the loss with respect to step S33. It can be appreciated that as the learning progresses, the loss for each step decreases.

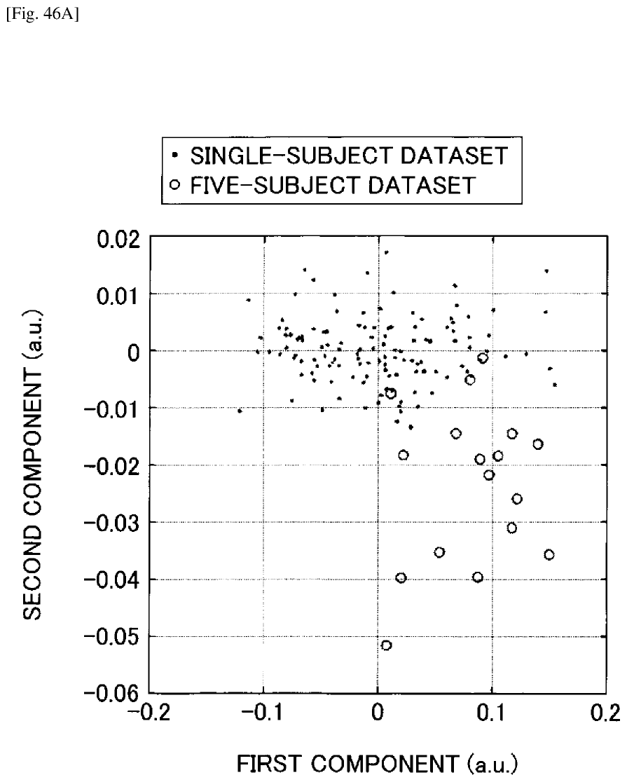

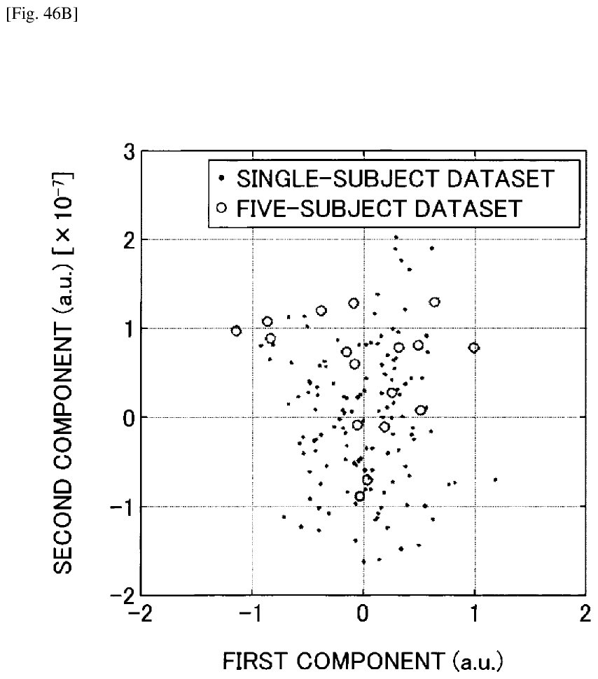

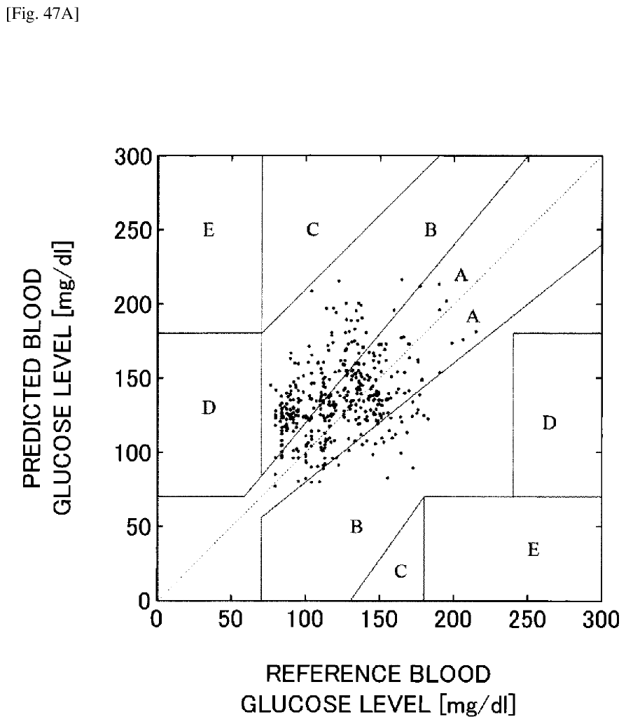

FIGS. 46A and 46B are graphs showing data distributions for a representative series of dataset 2 with and without domain adaptation (DA). FIG. 46A represents the distribution of input data input to layer L₁ (without DA). FIG. 46B represents the distribution of output data from layer L₃ (with DA). The fine dots represent data points of dataset 1 (supervised data), and the circle marks represent data points of dataset 2 (unsupervised data).

Both FIGS. 46A and 46B are plotted by reducing the three-dimensional data to two dimensions using principal component analysis. At the input stage as represented by FIG. 46A, the distribution of dataset 1 and the distribution of dataset 2 are substantially different. However, in FIG. 46B representing the output data from layer L₃, the distributions of dataset 1 and dataset 2 considerably overlap with each other. It can be appreciated from these findings that the network according to the present embodiment can absorb the differences between dataset 1 and dataset 2.

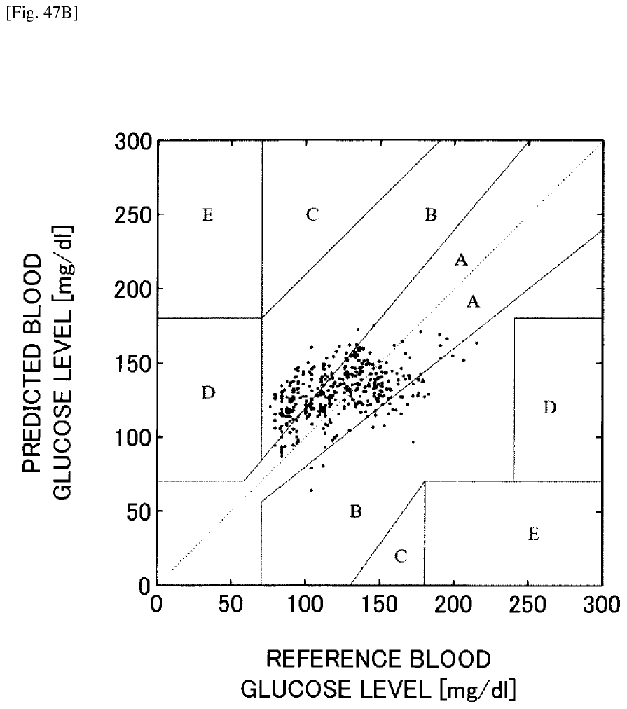

FIGS. 47A and 47B are Clarke error grids showing prediction accuracies of prediction models obtained with and without domain adaptation (DA). FIG. 47A is a Clarke error grid for dataset 2 when DA is not implemented and represents the prediction accuracy of a prediction model obtained from data of dataset 1 by executing only step S31 in FIG. 44. FIG. 47B is a Clarke error plot for dataset 2 when DA is implemented and represents the prediction accuracy of a prediction model obtained by executing steps S31 to S33 of FIG. 44.

For the prediction model obtained without DA, the correlation coefficient is 0.38, and 53.6% of the data points are included in region A of FIG. 47A. For the prediction model obtained with DA, the correlation coefficient is 0.47, and 63.8% of the data points are in regions A+B of FIG. 47B. It can be appreciated from the above comparison that by using the calibrator 455 according to the present embodiment, a higher correlation coefficient can be achieved and errors can be reduced. That is, by implementing domain adaptation, a prediction model can be appropriately calibrated without requiring blood sampling. Also, the test data used includes data of various circumstances in terms of meals, subjects, measurement temperature, and the like, and the fact that correlation can be found with respect to such unspecified data indicates that high generalization performance and robust measurement can be achieved.

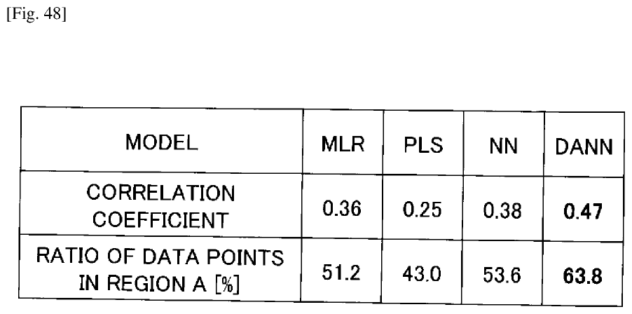

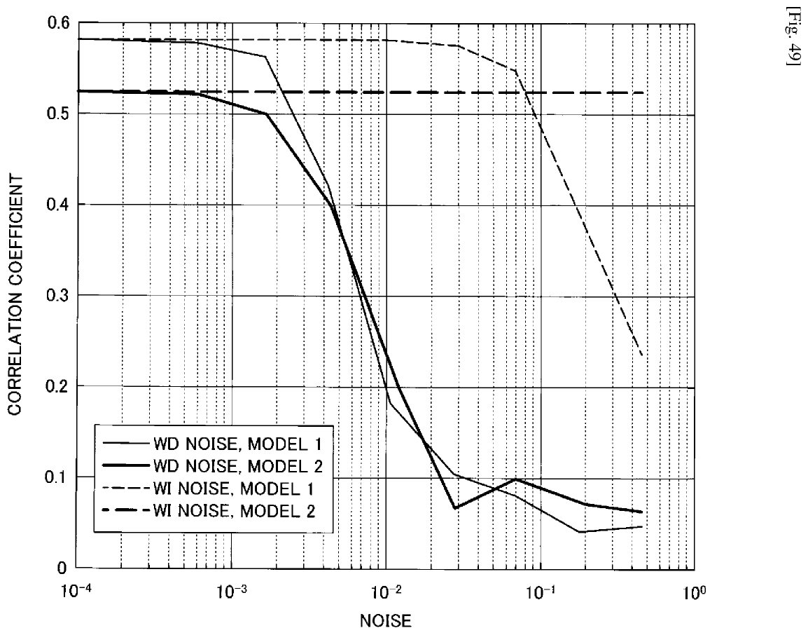

FIG. 48 is a table comparing the correlation coefficient and the ratio of data points included in region A of the Clarke error grid for the DANN using the calibrator 455 and various other models. Note that the table of FIG. 48 reflects the results obtained in FIGS. 11A to 12B for the MLR (multiple linear regression) model and the PLS (partial least-squares). FIG. 48 also indicates results of a neural network (NN) that does not implement domain adaptation and adversarial update.

Note that the above four models all share a common condition that calibration by blood sampling is not performed. In the models other than DANN, calibration is not performed with respect to each series of the five-subject dataset (dataset 2). Because the PLS model has a wavenumber selection function, its input is assumed to be a broad spectrum absorbance data (measured every 2 cm⁻¹ from 980 cm⁻¹ to 1200 cm⁻¹). The input wavenumbers for the models other than PLS are 1050 cm⁻¹, 1070 cm⁻¹, and 1100 cm⁻¹.

It has been shown that PLS, which is generally used for spectral analysis, does not give acceptable results without calibration. This is thought to be due to the fact that the number of wavenumbers of the input spectrum is larger than the number of data, such that performance is degraded by the influence of overfitting. Because the NN model can deal with nonlinear components, it is somewhat more accurate than MLR. DANN shows the best results among the tested techniques.

By using the calibrator 455 according to the present embodiment, blood sampling for calibration becomes unnecessary and obstacles associated with performing calibration can be reduced. Calibration may be automatically performed at the user site at the time of measurement, and measurement accuracy may be improved. Even when the measuring apparatus according to the present embodiment is applied to a simple monitoring apparatus for home use, for example, measurement accuracy may be substantially improved. The measuring apparatus and calibration method according to embodiments of the present invention are not limited to being applied to blood glucose level measurement, but may be applied to other various measurements that generally require calibration with respect to each individual that involves invasive procedures such as blood sampling.

<Influence of Light Source Noise on Prediction Model>

In the following, the influence of light source noise on the prediction model will be considered. When a plurality of lasers are used as light sources as illustrated in FIG. 39, for example, the influence of light source noise is preferably taken into consideration.

Wavenumbers to be selectively used for noninvasive blood glucose level measurement may include at least one of 1050±6 cm⁻¹, 1070±6 cm⁻¹, and 1100±6 cm⁻¹. For example the wavenumbers 1050 cm⁻¹, 1070 cm⁻¹, and 1100 cm⁻¹ may be used. Note that although a wavenumber other than the wavenumbers to be used for measurement is selectively used as a normalization wavenumber in the above-described embodiment, in other embodiments, one of the wavelengths to be used for measurement may be used for normalization.

As prediction models, a linear regression model (model 1) that uses three wavenumbers including 1050 cm⁻¹, 1070 cm⁻¹, and 1100 cm'; and a normalized linear regression model (model 2) that uses one of the above wavenumbers for normalization are used. In the present example, the wavenumber 1050 cm⁻¹ is used as the wavelength for normalization in the normalized linear regression model. However, any one of the above three wavenumbers may be set up as the denominator (wavenumber for normalization) of the normalized linear regression model without producing substantial differences in results.

In the case of using a quantum cascade laser (QCL) as the light source, in view of wavenumber deviations due to aspects of QCL fabrication, a QCL with an actual output of 1092 cm⁻¹ is contemplated for use as the light source for the above selected wavenumber 1100 cm⁻¹. That is, in the following description, prediction models using three wavenumbers including 1050 cm⁻¹, 1070 cm⁻¹, and 1092 cm⁻¹ are contemplated.

Model 1 (linear regression model) can be represented by the following equation (16).

[Math.20]

y=−1253·x(1050 cm⁻¹)+2159·x(1070 cm⁻¹)−1029·x(1092 cm⁻¹)+198 (16)

Model 2 (normalized linear regression model) can be represented by the following equation (17).

$$[ Math .21 ]$$

$$y = \frac{\begin{matrix}- 770 \cdot x ( 1050 {cm}^{- 1} ) + 1770 \cdot x ( 1070 {cm}^{- 1} ) - \\ 906 \cdot x ( 1092 {cm}^{- 1} )\end{matrix}}{x ( 1050 {cm}^{- 1} )} \qquad ( 17 )$$

In the above equations (16) and (17), x (λ) represents the absorbance at wavelength λ, and y represents the predicted value of the blood glucose level in blood. In both model 1 and model 2, all data of dataset 1 of FIG. 3 are learned to obtain regression coefficients of the prediction model.

As a noise model, two types of noise including wavelength dependent noise (or wavenumber dependent noise), referred to as “WDnoise”, and wavelength independent noise (or wavenumber independent noise), referred to as “WInoise”, may be contemplated. The noise model can be represented by the following equation (18).

[Math.22]

x_{N}(λ)=N_{WI}·N_{WD}(λ)·x(λ) (18)

In the above equation (18), x (λ) represents the absorbance measured at wavelength λ, and x_{N}(λ) represents the absorbance with noise added. N_{WI} represents the amount of wavelength independent noise (WInoise), and N_{WD}(λ) represents the amount of wavelength dependent noise (WDnoise). The wavelength dependent noise represents noise due to power fluctuations, wavelength fluctuations, polarization fluctuations of the QCL of each wavelength (wavenumber) and noise due to accompanying transmission line and ATR mode fluctuations. On the other hand, the wavelength independent noise represents noise due to factors that are considered independent of the wavelength, such as variations in the state of contact between the ATR optical element and the sample to be measured.

The above noise terms are defined by the following models.

N_{WI}=N(1, noise_{WI}²)

N_{WD}(λ)=N(1, noise_{WD}²) [Math.23]

Note that N(1, noise_{WI}²) and N(1, noise_{WD}²) of the above models respectively represent normal distributions with a mean of 1 and standard deviations of noiseWI and noiseWD.

As the evaluation method, a random number of the normal distribution is generated, and an input signal with noise added is simulated by calculating equation (18). Using the input signal, the correlation coefficient of the prediction result using each model is obtained by Monte Carlo simulation, and the correlation coefficient under each condition is regarded as a performance evaluation value. The number of iterations for each condition is 10, and the average value is regarded as the simulation result.

Simulations are performed with respect to each of the wavelength independent noise (WInoise) and the wavelength dependent noise (WDnoise) and with respect to each of model 1 and model 2. Also, simulations are performed with respect to each type of noise and with respect to each of dataset 1 and dataset 2. However, with regard to dataset 1, because dataset 1 is also used for parameter learning, it may be used as a reference value.

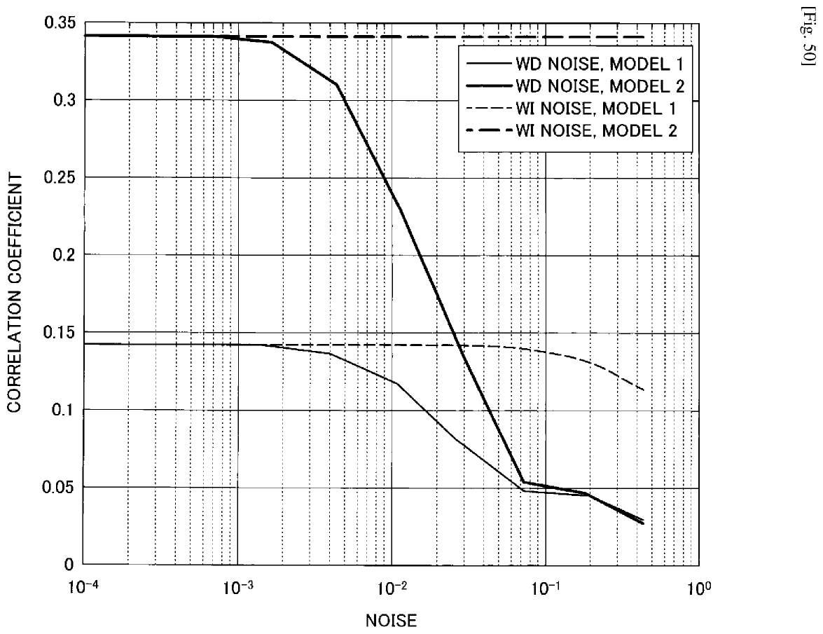

FIG. 49 shows the simulation results for dataset 1 and FIG. 50 shows the simulation results for dataset 2. In FIGS. 49 and 50, the horizontal axis represents noise and the vertical axis represents the correlation coefficient. With regard to wavelength independent noise (WInoise), because model 2 is normalized, model 2 is insensitive with respect to the amount of wavelength independent noise. Also, as can be appreciated from the simulation results for dataset 2 of FIG. 50, model 2 shows better results in terms of generalization performance as compared with model 1. That is, by using the prediction model 2 that is normalized using one wavenumber (wavelength) from among the wavenumbers (wavelengths) used, performance may be enhanced for unknown data. Also, even when the non-normalized model 1 is used, sensitivity for the wavelength independent noise (WInoise) is higher by at least one order of magnitude as compared with that for the wavelength dependent noise (WDnoise). That is, when the light source noise arranged to be wavelength independent noise (WInoise), measurement accuracy can be improved.

Note that because both dataset 1 and dataset 2 already have various types of noise (including WDnoise and WInoise) due to individual fluctuations, measurement time fluctuations with respect to FTIR, and the like, the correlation coefficients at the left side of the graphs of FIGS. 49 and 50 are saturated. Thus, regions in the graphs where the noise added in the simulation becomes dominant, i.e., the regions at the right side of the graphs where the correlation coefficients are decreasing, constitute effective prediction results of accuracy with respect to the amount of noise.

As for the amount of wavelength independent noise (WInoise), the simulation results for dataset 2 shown in FIG. 50 suggest that the allowed amount of variation is approximately 0.5% by standard deviation for achieving a correlation coefficient R greater than 0.3 (R>0.3). Although the simulation results for dataset 1 shown in FIG. 49 correspond to reference values used as learning data, the results suggest that the amount of variation has to be controlled to approximately 0.2% by standard deviation in order to achieve a correlation coefficient R greater than 0.5 (R>0.5).

Based on the above simulations, the allowed amount of variation in the wavelength independent noise for achieving a correlation coefficient R that is greater than 0.3 (R>0.3) is approximately 0.5% by standard deviation. In order to achieve a correlation coefficient R that is greater than 0.5 (R>0.5), the amount of variation is preferably controlled to approximately 0.2% by standard deviation. As for the prediction model, a normalized linear regression model rather than a general linear regression model is preferably used in view of its generalization performance and insensitivity to wavelength independent noise.

Although the present invention has been described with respect to illustrative embodiments, the present invention is not limited to these embodiments and numerous variations and modifications may be made without departing from the scope of the present invention.

The present application is based on and claims the benefit of the priority date of

Japanese Patent Application No. 2017-160481 filed on Aug. 23, 2017 and Japanese Patent Application No. 2018-099150 filed on May 23, 2018, the entire contents of which are hereby incorporated by reference.

1. A measuring apparatus comprising:
a light source configured to output light in a mid-infrared region;
a detector configured to irradiate a measuring object with the light output from the light source and detect reflected light reflected by the measuring object; and
a blood glucose level measuring device configured to measure a blood glucose level of the measuring object;
wherein a wavenumber between a plurality of absorption peak wavenumbers of glucose is used as a blood glucose level measuring wavenumber for measuring the blood glucose level.

2. The measuring apparatus according to claim 1, wherein the blood glucose level measuring wavenumber includes at least one wavenumber selected from a group consisting of a wavenumber between 1035 cm¹ and 1080 cm¹ and a wavenumber between 1080 cm⁻¹ and 1110 cm⁻¹.

3. The measuring apparatus according to claim 2, wherein the blood glucose level measuring wavenumber includes at least one wavenumber selected from a group consisting of 1050±6 cm⁻¹, 1070±6 cm⁻¹, and 1100±6 cm⁻¹.

4. The measuring apparatus according to claim 2, wherein the blood glucose level measuring wavenumber is a wavenumber that enables separation of an absorption spectrum of glucose from an absorption spectrum of a metabolite other than glucose.

5. The measuring apparatus according to claim 1, wherein
the blood glucose level measuring device determines the blood glucose level based on a prediction model generated from data normalized with respect to a wavenumber for normalization; and
the wavenumber for normalization is one wavenumber selected from the blood glucose level measuring wavenumber.

6. The measuring device according to claim 1, further comprising:
a reliability estimating device configured to estimate a reliability of measurement;
wherein the light source outputs light with a wavenumber for reliability estimation that is different from the blood glucose level measuring wavenumber; and
wherein the reliability estimating device estimates the reliability of measurement based on first data obtained using the blood glucose level measuring wavenumber and second data obtained using the wavenumber for reliability estimation.

7. The measuring apparatus according to claim 1, further comprising:
a calibrator configured to calibrate the blood glucose level measured by the blood glucose level measuring device; and
a memory storing first spectrum data including blood glucose level label information;
wherein the calibrator acquires second spectrum data at the blood glucose level measuring wavenumber that does not include the blood glucose level label information and combines the first spectrum data and the second spectrum data to generate a prediction model.

8. The measuring apparatus according to claim 7, wherein the prediction model includes a domain adaptation function.

9. The measuring apparatus according to claim 8, wherein the prediction model is generated using an output of a discriminator that discriminates between the first spectrum data and the second spectrum data.

10. The measuring apparatus according to claim 9, wherein the calibrator updates learning of the prediction model such that the first spectrum data and the second spectrum data cannot be discriminated based on the output of the discriminator.

11. A measuring method comprising:
irradiating a measuring object with light in a mid-infrared region output from a light source;
detecting an absorption spectrum of reflected light reflected by the measuring object; and
measuring a blood glucose level of the measuring object based on the absorption spectrum;
wherein a wavenumber between a plurality of absorption peak wavenumbers of glucose is used as a blood glucose level measuring wavenumber for measuring the blood glucose level.

12. The measuring method according to claim 11, wherein the blood glucose level measuring wavenumber includes at least one wavenumber selected from a group consisting of a wavenumber between 1035 cm⁻¹ and 1080 cm⁻¹ and a wavenumber between 1080 cm¹ and 1110 cm⁻¹.

13. The measuring method according to claim 12, wherein the blood glucose level measuring wavenumber includes at least one wavenumber selected from a group consisting of 1050±6 cm¹, 1070±6 cm⁻¹, and 1100±6 cm⁻¹.

14. The measuring method according to claim 11, further comprising:
acquiring first spectrum data including blood glucose level label information;
acquiring second spectrum data at the blood glucose level measuring wavenumber that does not include the blood glucose level label information; and
combining the first spectrum data and the second spectrum data to generate a prediction model for regressing measured spectrum data to the blood glucose level.

15. The measuring method according to claim 14, further comprising:
generating the prediction model from data normalized with respect to a wavenumber for normalization corresponding to one wavenumber selected from the blood glucose level measuring wavenumber; and
determining the blood glucose level based on the prediction model.
